(19) United States
(12) Patent Application Publication
(10) Pub. No.: US 2023/0348779 A1
(43) Pub. Date: Nov. 2, 2023

(54) FLUORESCENT COMPOUND AND FLUORESCENTLY LABELED BIOLOGICAL SUBSTANCE USING THE SAME

(71) Applicant: FUJIFILM Corporation, Tokyo (JP)

(72) Inventors: Masato SAKAKIBARA, Kanagawa (JP); Hiyoku NAKATA, Kanagawa (JP); Hiroaki TANAKA, Kanagawa (JP)

(73) Assignee: FUJIFILM Corporation, Tokyo (JP)

(21) Appl. No.: 18/347,582

(22) Filed: Jul. 6, 2023

Related U.S. Application Data
(63) Continuation of application No. PCT/JP2022/009678, filed on Mar. 7, 2022.

(30) Foreign Application Priority Data
Mar. 8, 2021 (JP) 2021-036777

(51) Int. Cl. C09K 11/06; G01N 33/533

(52) U.S. Cl. CPC C09K 11/06; G01N 33/533; C09K 2211/1096

(57) ABSTRACT

There are provided a fluorescent compound of the following formula or a salt thereof and a labeled biological substance.

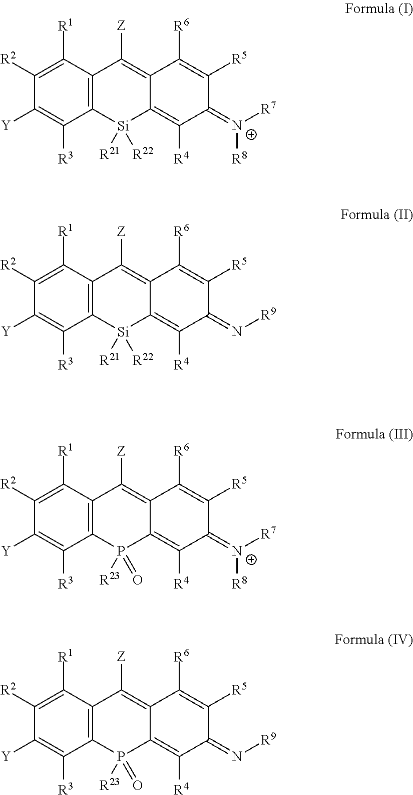

Y represents —NR¹⁰R¹¹ or —OR¹², and R¹ to R¹², R²¹ to R²³, and Z represent specific groups and satisfy the following regulations.
At least one of R²¹ or R²² is an alkenyl group, an alkynyl group, an aryl group, or a heteroaryl group. In a case of R²¹=an aryl group and R²²=an alkyl group or an aryl group, at least R⁷ and R⁸, or R¹⁰ and R¹¹ are bonded to each other to form a 4-membered ring.
A compound of Formula (I) or (II) or a salt thereof satisfies condition α, and a compound of Formula (III) or (IV) or a salt thereof satisfies condition β.
(Condition α)
In a case where Z is an aryl group, the aryl group has a specific structure.
(Condition β)
At least R⁷ and R⁸, or R¹⁰ and R¹¹ are bonded to each other to form a 4-membered ring.

### CROSS-REFERENCE TO RELATED APPLICATIONS

This application is a Continuation of PCT International Application No. PCT/JP2022/009678 filed on Mar. 7, 2022, which claims priority under 35 U.S.C. § 119 (a) to Japanese Patent Application No. 2021-036777 filed in Japan on Mar. 8, 2021. Each of the above applications is hereby expressly incorporated by reference, in its entirety, into the present application.

### BACKGROUND OF THE INVENTION

### 1. Field of the Invention

The present invention relates to a fluorescent compound and a fluorescently labeled biological substance using the fluorescent compound.

### 2. Description of the Related Art

In pathological examinations, identification of infectious diseases, and the like, fluorescently labeled biological substances obtained by labeling biological substances such as an antibody that binds to a detection target substance of interest, with a compound (a dye) are used.

Among them, bioimaging technology for analyzing the dynamics and functions of living body molecules, cells, tissues, and the like in the living body has been utilized for the diagnosis of various diseases. In recent years, in vivo fluorescence imaging, which visualizes and observes a specific portion of a living body with a fluorescent dye, is expected as a new technique for living body observation.

In this in vivo fluorescence imaging, an organic fluorescent dye is generally used. However, the organic fluorescent dye has low light resistance and deteriorates by irradiation with excitation light, and thus the observation of the living body of interest may not be sufficiently carried out.

Rhodamine is known as a fluorescent dye having a high fluorescence quantum yield and high light resistance (photofading resistance) and is used as a dye in a fluorescently labeled biological substance.

On the other hand, as described in WO2020/033681A, WO2014/106957A, WO2014/144793A, US2018/0284105A, US2019/0100653A, U.S. Pat. No. 8,506,655B, and WO2018/043579A, research on fluorescent dyes in which the absorption wavelength and the fluorescence wavelength are lengthened by replacing an oxygen atom which is a ring-constituting atom of a xanthene skeleton of rhodamine with a silicon atom or a phosphorus atom is also underway.

### SUMMARY OF THE INVENTION

However, as a result of studies by the inventors of the present invention, it was found that it cannot be said that the rhodamine compounds as described in Patent WO2020/033681A, WO2014/106957A, WO2014/144793A, US2018/0284105A, US2019/0100653A, U.S. Pat. No. 8,506,655B, and WO2018/043579A are sufficient from the viewpoint of achieving both an excellent fluorescence quantum yield and an excellent light resistance.

In addition, regarding the compound as well, in which an oxygen atom which is a ring-constituting atom of a xanthene skeleton contained in rhodol with a silicon atom or a phosphorus atom, there is also a demand for a compound having both an excellent fluorescence quantum yield and an excellent light resistance.

An object of the present invention is to provide rhodamine or a rhodol compound, in which an oxygen atom which is a ring-constituting atom of a xanthene ring is replaced with a silicon atom or a phosphorus atom, where it is a fluorescent compound capable of achieving both an excellent fluorescence quantum yield and an excellent light resistance. In addition, another object of the present invention is to provide a fluorescently labeled biological substance obtained by bonding the fluorescent compound to a biological substance.

That is, the above objects of the present invention have been achieved by the following means.

[1] A fluorescent compound represented by any one of General Formulae (I) to (IV) or a salt thereof,

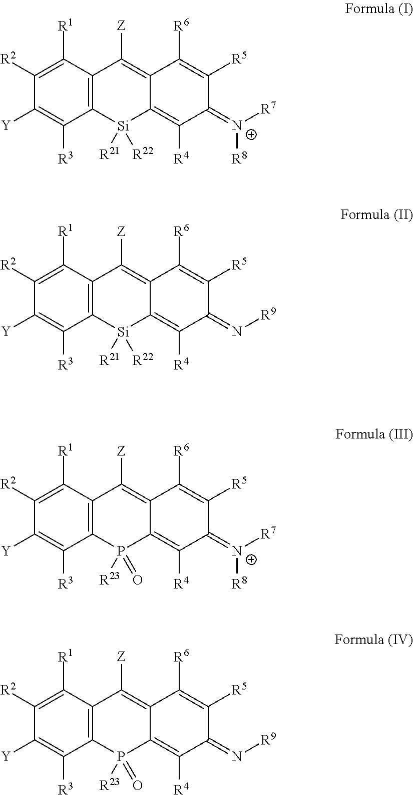

in the formulae, Y represents —NR¹⁰R¹¹ or —OR¹², and Z represents a group represented by Formula (A),
R¹ to R⁶ represent a halogen atom, a cyano group, or a group represented by Formula (A),
R⁷ to R¹² represent a group represented by Formula (A), where R⁷ and R⁸ may be bonded to each other to form a 4- to 7-membered aliphatic heterocyclic ring, and R¹⁰, R¹¹ may be bonded to each other to form a 4- to 7-membered aliphatic heterocyclic ring, and R⁷ to R¹¹ each may be bonded to adjacent R² to R⁵ to form a 5- to 7-membered aliphatic heterocyclic ring or aromatic heterocyclic ring,
R²¹ and R²² represent a group represented by Formula (A), and R²¹ and R²² may be bonded to each other to form a 4- to 7-membered aliphatic heterocyclic ring, provided that at least one of R²¹ or R²² is an alkenyl group, an alkynyl group, an aryl group, or a heteroaryl group, and in a case where R²¹ is an aryl group and R²² is an alkyl group or an aryl group, in at least one of a combination of R⁷ and R⁸ or a combination of R¹⁰ and R¹¹, R⁷ and R⁸, or R¹⁰ and R¹¹ are bonded to each other to form a 4-membered aliphatic heterocyclic ring, and
R²³ represents a group represented by Formula (A),

*-L³-R¹¹¹ Formula (A)
in the formula, L³ is a single bond or a linking group obtained by combining one or two or more kinds among an alkylene group, an alkenylene group, an alkynylene group, an arylene group, a heteroarylene group, and each group represented by any one of Formula (1-1) to Formula (1-8),
R¹¹¹ represents a hydrogen atom, an alkyl group, an alkenyl group, an alkynyl group, an aryl group, a heteroaryl group, or a monovalent aliphatic heterocyclic group,
provided that in a case where a hydrogen atom as R¹¹¹ is a dissociative hydrogen atom, this dissociative hydrogen atom may be dissociated, and
* represents a bonding portion,

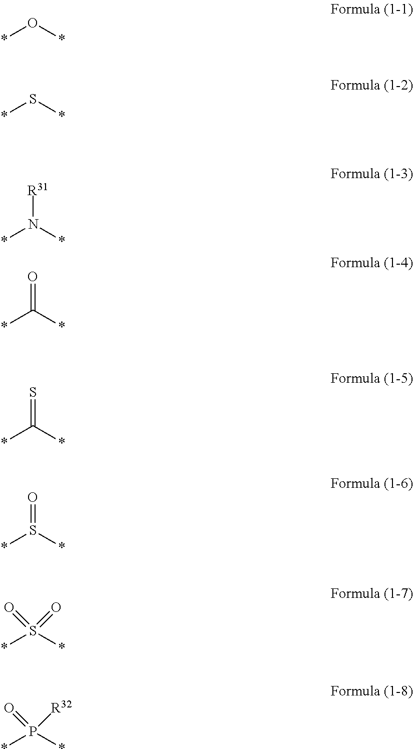

in the formulae, R³¹ and R³² represent a hydrogen atom or a substituent, and
* represents a bonding portion,
provided that the compound represented by Formula (I) or (II) or a salt thereof satisfies the following condition α, and the compound represented by Formula (III) or (IV) or a salt thereof satisfies the following condition β,

(The Condition α)
in a case where Z is an aryl group, this aryl group is a group represented by Formula (C),

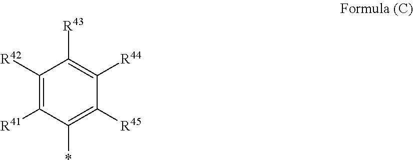

in the formula, R⁴¹ to R⁴⁵ represent a hydrogen atom or a substituent, and * represents a bonding portion,
provided that at least one of (C-1), (C-2), or (C-3) is satisfied,
(C-1) at least one of R⁴², R⁴³, or R⁴⁴ is a group having any of a carboxy group or a substituent capable of being bonded to a biological substance,
(C-2) R⁴¹ and R⁴⁵ are an alkyl group or a halogen atom,
(C-3) at least one of R⁴¹ or R⁴⁵ is a fluorine atom or an alkoxy group,

(The Condition β)
in at least one of a combination of R⁷ and R⁸ or a combination of R¹⁰ and R¹¹, R⁷ and R⁸, or R¹⁰ and R¹¹ are bonded to each other to form a 4-membered aliphatic heterocyclic ring.

[2] The fluorescent compound or a salt thereof according to [1], in which the fluorescent compound or a salt thereof is represented by General Formula (I) or (II).

[3] The fluorescent compound or a salt thereof according to [2], in which in at least one of the combination of R⁷ and R⁸ or the combination of R¹⁰ and R¹¹, R⁷ and R⁸, or R¹⁰ and R¹¹ are bonded to each other to form a 4- to 7-membered aliphatic heterocyclic ring, provided that in a case where R²¹ is an aryl group and R²² is an alkyl group or an aryl group, the ring formed by bonding R⁷ and R⁸, or R¹⁰ and R¹¹ to each other in at least one of the combination of R⁷ and R⁸ or the combination of R¹⁰ and R¹¹ is a 4-membered aliphatic heterocyclic ring.

[4] The fluorescent compound or a salt thereof according to [2] or [3], in which at least one of R⁷, . . . , or R¹¹ is a group including an electron withdrawing group.

[5] The fluorescent compound or a salt thereof according to any one of [2] to [4], in which Z is a group represented by Formula (C).

[6] The fluorescent compound or a salt thereof according to any one of [2] to [5], in which Y is —NR¹⁰R¹¹.

[7] The fluorescent compound or a salt thereof according to any one of [2] to [6], in which the fluorescent compound or a salt thereof is represented by General Formula (IA) or (IIA),

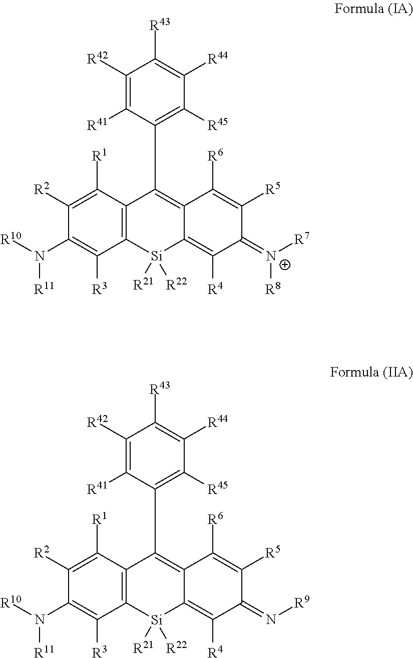

in the formulae, R¹ to R¹¹ and R⁴¹ to R⁴⁵ respectively have the same meanings as R¹ to R¹¹ and R⁴¹ to R⁴⁵,

R²¹ and R²² represent the group represented by Formula (A), and R²¹ and R²² may be bonded to each other to form a 4- to 7-membered aliphatic heterocyclic ring, provided that at least one of R²¹ or R²² is an alkenyl group or an aryl group, and in a case where R²¹ is an aryl group and R²² is an alkyl group or an aryl group, in at least one of the combination of R⁷ and R⁸ or the combination of R¹⁰ and R¹¹, R⁷ and R⁸, or R¹⁰ and R¹¹ are bonded to each other to form a 4-membered aliphatic heterocyclic ring.

[8] The fluorescent compound or a salt thereof according to [7], in which the fluorescent compound or a salt thereof is represented by General Formula (IA), R⁷, R⁸, R¹⁰, and R¹¹ are a hydrogen atom or an alkyl group having 1 to 3 carbon atoms, and R⁴¹ to R⁴⁵ satisfy (C-1) and (C-2), satisfy (C-1) and (C-3), or satisfy all of (C-1) to (C-3) in the condition α.

[9] The fluorescent compound or a salt thereof according to [7], in which the fluorescent compound or a salt thereof is represented by General Formula (IA), in at least one of the combination of R⁷ and R⁸ or the combination of R¹⁰ and R¹¹, R⁷ and R⁸, or R¹⁰ and R¹¹ are bonded to each other to form a 4- to 7-membered aliphatic heterocyclic ring, and R⁴¹ to R⁴⁵ satisfy (C-1) and (C-2), satisfy (C-1) and (C-3), or satisfy all of (C-1) to (C-3) in the condition α.

[10] The fluorescent compound or a salt thereof according to [1], in which the fluorescent compound or a salt thereof is represented by General Formula (III) or (IV).

[11] The fluorescent compound or a salt thereof according to [10], in which at least one of R⁷, . . . , or R¹¹ is a group including an electron withdrawing group.

[12] The fluorescent compound or a salt thereof according to [10] or [11], in which R²³ is a hydroxy group, an alkoxy group, an aryl group, or an alkyl group, provided that in a case where R²³ is a hydroxy group, a hydrogen atom in this hydroxy group may be dissociated.

[13] A fluorescently labeled biological substance that is obtained by bonding a biological substance to the fluorescent compound or a salt thereof according to any one of [1] to [12].

[14] The fluorescently labeled biological substance according to [13], in which the biological substance is any one of a protein, an amino acid, a nucleic acid, a sugar chain, or a lipid.

[15] The fluorescently labeled biological substance according to [13] or [14], in which the bonding between the fluorescent compound or a salt thereof and the biological substance is a bonding formed by any one of the following i) to v),
i) a non-covalent bond or covalent bond between peptides,
ii) a Van der Waals interaction between a long-chain alkyl group in a fluorescent compound and a lipid bilayer or lipid in a biological substance,
iii) an amide bond formed by reacting an N-hydroxysuccinimide ester in a fluorescent compound with an amino group in a biological substance,
iv) a thioether bond formed by reacting a maleimide group in a fluorescent compound with a sulfanyl group in a biological substance,
v) a bond associated with a formation of a triazole ring, which is formed by a Click reaction between an azido group in a fluorescent compound and an acetylene group in a biological substance, or between an acetylene group in a fluorescent compound and an azido group in a biological substance.

The fluorescent compound according to the aspect of the present invention is a compound capable of achieving both an excellent fluorescence quantum yield and an excellent light resistance. In addition, the fluorescently labeled biological substance according to the aspect of the present invention can be obtained by using a fluorescent compound having an excellent fluorescence quantum yield and excellent light resistance.

### DESCRIPTION OF THE PREFERRED EMBODIMENTS

In the present invention, in a case where there are a plurality of substituents or linking groups represented by a specific symbol or formula (hereinafter, referred to as substituents or the like), or in a case where a plurality of substituents or the like are regulated at the same time, the substituents or the like may be the same or different from each other, unless otherwise specified. The same applies to the regulation of the number of substituents or the like. Further, in a case where a plurality of substituents or the like come close to each other (particularly in a case where they are adjacent to each other), they may be linked to each other to form a ring, unless otherwise specified. Further, unless otherwise specified, rings such as an alicyclic ring, an aromatic ring, and a heterocycle may be fused to form a fused ring.

In the present invention, in a case where E type and Z type of the double bond are present in the molecule, any one of the E type or the Z type, or a mixture thereof may be used unless otherwise specified. For example, a compound represented by any one of General Formulae (II) or (IV) is specifically shown for convenience as a structure in which a substituent R⁹ on the nitrogen atom and the carbon atom to which R⁵ is bonded are positioned on the same side with respect to the double bond. However, a structure in which a substituent R⁹ on the nitrogen atom and the carbon atom to which R⁵ is bonded are positioned on a side different from each other with respect to the double bond may be good, and these structures may be mixed, where a compound in which these structures are mixed is included in the compound represented by any one of General Formula (II) or (IV). In addition, in a case where a compound has a diastereomer and an enantiomer, any one of the diastereomer or the enantiomer may be used, or a mixture thereof may be used unless otherwise specified.

In the present invention, the representation of a compound or substituent is used to have a meaning including not only the compound itself but also a salt thereof, and an ion thereof. For example, in a group having a dissociative hydrogen atom, such as a carboxy group, a sulfo group, or a hydroxy group that can be adopted as R²³, the hydrogen atom may be dissociated so that the group has a corresponding ionic structure or salt structure.

In a case of a salt structure, the kind of the salt may be one kind, two or more kinds may be mixed, the group having the salt form and the group having the free acid structure may be mixed in the compound, and the compound having the salt structure and the compound having the free acid structure compound may be mixed.

The “salt” is meant to include a form in which a salt is formed in the molecule of the fluorescent compound according to the embodiment of the present invention. Examples of the salt of the carboxy group, the sulfo group, or the hydroxy group, which can be adopted as R²³ include salts of alkali metals such as Na, Li, and K, salts of alkaline earth metals such as Mg, Ca, and Ba, and salts of organic amines such as tetraalkylammonium. In addition, for example, a form in which the carboxy group, the sulfo group, or the hydroxy group, which can be adopted as R²³, has an ionic structure and the ionic structure forms a salt in the molecule together with the positively charged nitrogen atom to which R⁷ and R⁸ are bonded is included.

The “ion” means a form that is present as a negatively charged or positively charged atom or atomic group.

In the compound according to the embodiment of the present invention, for example, the compound represented by General Formula (I) or (III), the positive charge possessed by the compound is specifically shown for convenience as a structure possessed by the specific nitrogen atom. However, since the compound according to the embodiment of the present invention has a conjugated system, another atom other than the nitrogen atom actually may be capable of being positively charged, and thus any compound capable of adopting a structure represented by General Formula (I) or (III) as one of the chemical structures is included in the compound represented by General Formula (I) or (III). The same applies to the negative charge. The same applies to a compound represented by each of the other general formulae.

In addition, it is meant to include those in which a part of the structure is changed within the scope that does not impair the effects of the present invention. Furthermore, it is meant that a compound, which is not specified to be substituted or unsubstituted, may have any substituent within the scope that does not impair the effect of the present invention. The same applies to a substituent (for example, a group represented by “alkyl group”, “methyl group”, “methyl”) and a linking group (for example, a group represented by “alkylene group”, “methylene group”, “methylene”). Among such substituents, a preferred substituent in the present invention is a substituent selected from a substituent group T described later.

In the present invention, the rhodamine compound means a rhodamine compound in which the 3-position and the 6-position of the xanthene ring are substituted with an amino group, and the rhodol structure means a rhodol compound in which the 3-position or the 6-position of the xanthene ring is substituted with an amino group.

In addition, in the present invention, the Si-rhodamine compound or the Si-rhodol compound means a rhodamine compound or a rhodol compound, in which an oxygen atom which is a ring-constituting atom of a xanthene skeleton is replaced with a silicon atom, and the P-rhodamine compound or the P-rhodol compound means a rhodamine compound or a rhodol compound, in which an oxygen atom which is a ring-constituting atom of a xanthene skeleton is replaced with a phosphorus atom.

Further, in the compound according to the embodiment of the present invention, adjacent substituents among R¹ to R⁹, Y, and Z may be bonded to each other to form a ring, thereby forming a fused-ring structure. The number of rings to be formed is not particularly limited as long as it can be structurally adopted, and a plurality of rings may be formed.

In the present invention, in a case where the number of carbon atoms of a certain group is regulated, this number of carbon atoms means the number of carbon atoms of the entire group thereof unless otherwise specified in the present invention or the present specification. That is, in a case where this group has a form of further having a substituent, it means the total number of carbon atoms, to which the number of carbon atoms of this substituent is included.

In addition, in the present invention, the numerical range indicated by using “to” means a range including the numerical values before and after “to” as the lower limit value and the upper limit value, respectively.

<Fluorescent Compound Represented by any One of General Formulae (I) to (IV)>

The fluorescent compound represented by any one of General Formulae (I) to (IV) or a salt thereof according to the embodiment of the present invention is as follows and emits fluorescence. Hereinafter, the fluorescent compound represented by any one of General Formulae (I) to (IV) or a salt thereof according to the embodiment of the present invention will be simply referred to as a fluorescent compound according to the embodiment of the present invention.

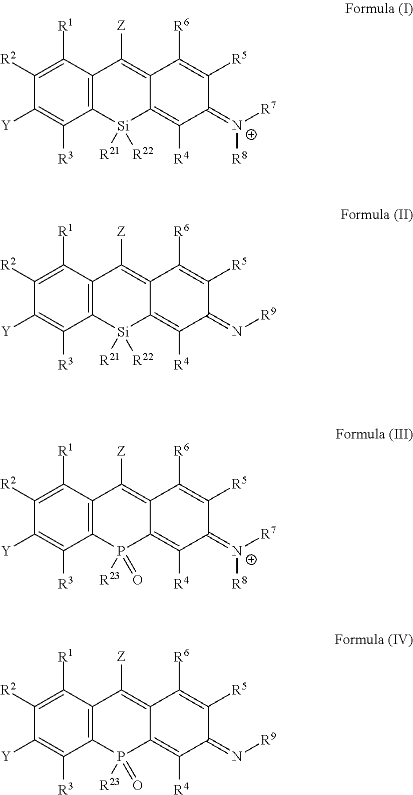

In the formulae, Y represents —NR¹⁰R¹¹ or —OR¹², and Z represents a group represented by Formula (A).

R¹ to R⁶ represent a halogen atom, a cyano group, or a group represented by Formula (A).

R⁷ to R¹² represent a group represented by Formula (A). Here, R⁷ and R⁸ may be bonded to each other to form a 4- to 7-membered aliphatic heterocyclic ring, and R¹⁰ and R¹¹ may be bonded to each other to form a 4- to 7-membered aliphatic heterocyclic ring. In addition, R⁷ to R¹¹ each may be bonded to adjacent R² to R⁵ to form a 5- to 7-membered aliphatic heterocyclic ring or aromatic heterocyclic ring.

R²¹ and R²² represent a group represented by Formula (A), and R²¹ and R²² may be bonded to each other to form a 4- to 7-membered aliphatic heterocyclic ring. However, at least one of R²¹ or R²² is an alkenyl group, an alkynyl group, an aryl group, or a heteroaryl group. In addition, in a case where R²¹ is an aryl group and R²² is an alkyl group or an aryl group, in at least one of a combination of R⁷ and R⁸ or a combination of R¹⁰ and R¹¹, R⁷ and R⁸, or R¹⁰ and R¹¹ are bonded to each other to form a 4-membered aliphatic heterocyclic ring.

R²³ represents a group represented by Formula (A).

However, the compound represented by Formula (I) or (II) or a salt thereof satisfies the condition α described later, and the compound represented by Formula (III) or (IV) or a salt thereof satisfies the condition β described later.

Hereinafter, the substituent and the like in Formula (I) to (IV) will be described in detail.

(Group Represented by Formula (A))

*-L³-R¹¹¹ Formula (A)

In the formula, L³ is a single bond or a linking group obtained by combining one or two or more kinds among an alkylene group, an alkenylene group, an alkynylene group, an arylene group, a heteroarylene group, and each group represented by any one of Formula (1-1) to Formula (1-8).

R¹¹¹ represents a hydrogen atom, an alkyl group, an alkenyl group, an alkynyl group, an aryl group, a heteroaryl group, or a monovalent aliphatic heterocyclic group.

However, in a case where a hydrogen atom as R¹¹¹ is a dissociative hydrogen atom, this dissociative hydrogen atom may be dissociated.

In addition, each group in L³ and R¹¹¹ may further have a substituent.

* represents a bonding portion.

It is noted that regarding the group represented by Formula (A), L³ and R¹¹¹ shall be interpreted based on the following rules (i) and (ii).

(i) A Case where the Group Represented by Formula (A) has a Group Represented by any One of General Formula (1-1) to (1-8)

A portion from * (bonding portion) to a group represented by any one of General Formula (1-1) to (1-8) is interpreted as L³.

For example, in a case where the group represented by Formula (A) is a carboxyphenyl group, the group represented by Formula (A) is a group represented by L³: a linking group obtained by combining a phenylene group, a group represented by Formula (1-4), and a group represented by Formula (1-1), and R¹¹¹: a hydrogen atom. In addition, in a case where a group represented by Formula (A) is a sulfoalkyl group, the group represented by Formula (A) is a group represented by L³: a linking group obtained by combining an alkylene, a group represented by Formula (1-7), and a group represented by Formula (1-1), and R¹¹¹: a hydrogen atom.

However, the group represented by any one of General Formulae (1-1) to (1-8) does not form a ring as ring-constituting atoms.

It is noted that in a case where the group represented by Formula (A) includes two or more groups represented by any of General Formula (1-1) to (1-8), a portion in the group represented by Formula (A) from * (bonding portion) to a group represented by any one of General Formulae (1-1) to (1-8) is interpreted as L³, where the group is positioned at a position where the number of shortest-bonded atoms from * (bonding portion) to the group represented by any one of General Formulae (1-1) to (1-8) is maximum.

(ii) A Case where the Group Represented by Formula (A) does not a Group Represented by any One of General Formula (1-1) to (1-8)

An alkyl group, an alkenyl group, an alkynyl group, an aryl group, a heteroaryl group, or a monovalent aliphatic heterocyclic group, which is positioned at the terminal structure in the group represented by Formula (A) is interpreted as R¹¹¹.

For example, in a case where the group represented by Formula (A) is a phenyl group, the group represented by Formula (A) is a group represented by L³: a single bond, and R¹¹¹: a phenyl group. In addition, in a case where the group represented by Formula (A) is —CH₂═CHPh, the group represented by Formula (A) is a group represented by L³: —CH₂═CH—, and R¹¹¹: Ph. Here, Ph means a phenyl group.

However, the “terminal structure of group represented by Formula (A)” means a structure located at a position closest to the terminal in the longest bonded chain in the group represented by Formula (A) in a case of being counted from * (the bonding portion).

In the above (i) and (ii), in a case where the group represented by Formula (A) is a group having a substituent capable of being bonded to a biological substance described later, it is read regarding a group in which, in the group represented by Formula (A), a substituent capable of being bonded to a biological substance described later is replaced with a hydrogen atom. That is, the substituent capable of being bonded to a biological substance described later is interpreted as a substituent which may be contained in each group as L³ or R¹¹¹.

In addition, each group defined by R¹¹¹ or L³ may further have a substituent within a range according to the above (i) and (ii).

(L³)

The alkylene group that can be adopted as L³ is synonymous with the group in which one hydrogen atom is further removed from the alkyl group selected from the substituent group T, which is described later, and the same applies to the preferred one thereof.

The alkenylene group that can be adopted as L³ is synonymous with the group in which one hydrogen atom is further removed from the alkenyl group selected from the substituent group T, which is described later, and the same applies to the preferred one thereof.

The alkynylene group that can be adopted as L³ is synonymous with the group in which one hydrogen atom is further removed from the alkynyl group selected from the substituent group T, which is described later, and the same applies to the preferred one thereof.

The arylene group that can be adopted as L³ is synonymous with the group in which one hydrogen atom is further removed from the aryl group selected from the substituent group T, which is described later, and the same applies to the preferred one thereof.

The heteroarylene group that can be adopted as L³ is synonymous with the group in which one hydrogen atom is further removed from the heteroaryl group selected from the substituent group T, which is described later, and the same applies to the preferred one thereof.

The alkylene group, the alkenylene group, the alkynylene group, the arylene group, and the heteroarylene group, which can be adopted as L³, may be an unsubstituted group or a group having a substituent.

The substituent which may be contained in the alkylene group, the alkenylene group, the alkynylene group, the arylene group, and the heteroarylene group, which can be adopted as L³, is not particularly limited, and it is preferably selected from a substituent group T described later. More preferred examples thereof include a halogen atom, an alkyl group, and an alkoxy group.

Examples of the halogen atom include a fluorine atom, a chlorine atom, a bromine atom, and an iodine atom, where a fluorine atom or a chlorine atom is preferable.

Further, the number of substituents that may be contained in the alkylene group, the alkenylene group, the alkynylene group, the arylene group, and the heteroarylene group, which can be adopted as L³, is not particularly limited as long as it can be adopted for the structure, and it can be one or more. The upper limit value thereof is not particularly limited, and for example, all hydrogen atoms in the alkylene group, the alkenylene group, the alkynylene group, the arylene group, and the heteroarylene group may be substituted with a substituent.

In the linking group obtained by combining one or two or more kinds among an alkylene group, an alkenylene group, an alkynylene group, an arylene group, a heteroarylene group, and each group represented by any one of Formula (1-1) to Formula (1-8), which can be adopted as L³, the kind of group to be combined is not particularly limited as long as the group to be combined has a reasonable chemical structure. For example, L³ does not include a group in which two or more groups each represented by any one of Formulae (1-1) to (1-3) are consecutive.

In the linking group obtained by combining one or two or more kinds among an alkylene group, an alkenylene group, an alkynylene group, an arylene group, a heteroarylene group, and each group represented by any one of Formula (1-1) to Formula (1-8), which can be adopted as L³, the kinds of groups to be combined are not particularly limited; however, for example, 1 to 6 kinds are preferable, and 1 to 4 kinds are more preferable.

In the linking group obtained by combining one or two or more kinds among an alkylene group, an alkenylene group, an alkynylene group, an arylene group, a heteroarylene group, and each group represented by any one of Formula (1-1) to Formula (1-8), which can be adopted as L³, the number of groups to be combined is not particularly limited; however, preferred examples thereof include 1 to 10, where 1 to 6 is more preferable, and 1 to 4 is still more preferable.

The groups represented by any of Formula (1-1) to (1-8), which can be adopted as L³, are as follows.

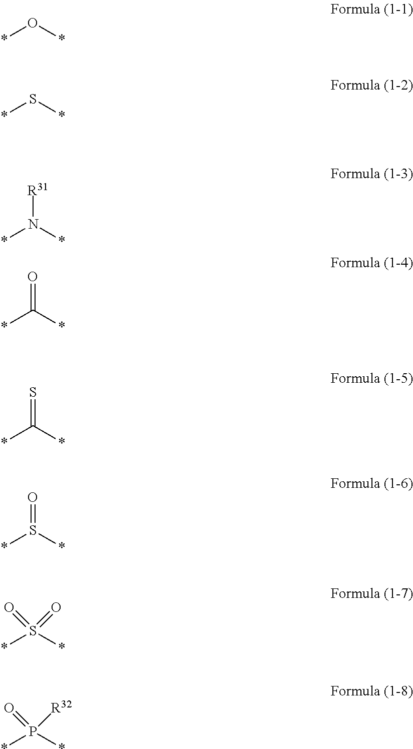

In the formulae, R³¹ and R³² represent a hydrogen atom or a substituent.

* represents a bonding portion.

The substituent that can be adopted as R³¹ is not particularly limited and is preferably selected from the substituent group T, which is described later. R³¹ is preferably a hydrogen atom, an alkyl group, an aryl group, a heteroaryl group, an acyl group, or a sulfonyl group, more preferably a hydrogen atom or an alkyl group, and still more preferably a hydrogen atom.

All the alkyl group, aryl group, heteroaryl group, acyl group, and sulfonyl group that can be adopted as R³¹ may have no substituent or may have a substituent.

The substituent that can be adopted as R³² is not particularly limited and is preferably selected from the substituent group T, which is described later. R³² is preferably a hydrogen atom, a hydroxy group, an alkoxy group, an aryloxy group, an alkyl group, an aryl group, or a heteroaryl group, more preferably a hydroxy group, an alkoxy group, or an aryloxy group, and still more preferably a hydroxy group.

Preferred examples of the group obtained by combining groups represented by any of Formulae (1-1) to (1-8) include groups represented by any of Formulae (1A-1) to (1A-9).

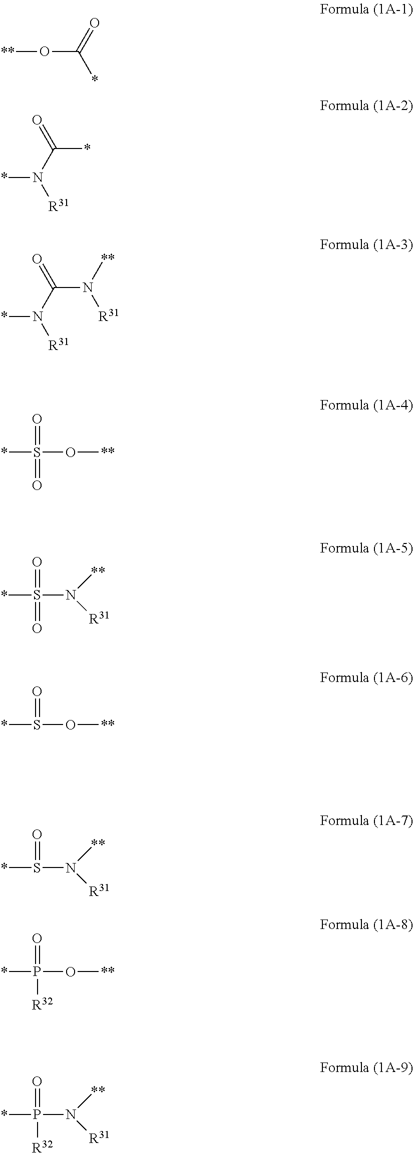

R³¹ and R³² respectively are synonymous with the above R³¹ and R³².

* and ** represent a bonding portion. It is noted that ** represents a bonding portion to R¹¹¹ side in a case where the group is a group that can be adopted as L³. Formula (1A-2) may be bonded to R¹¹¹ on any * side in L³.

The linking group obtained by combining each group represented by any one of Formulae (1-1) to (1-8), which can be adopted as the above L³, is preferably a group represented by any of Formula (1A-1), (1A-2), or (1A-4), more preferably a group represented by Formula (1A-1) or (1A-2), and still more preferably a group represented by Formula (1A-1).

For example, a group represented by the group represented by Formula (1A-1) and the group represented by the hydrogen atom as R¹¹¹ corresponds to a carboxy group, and a group represented by the group represented by Formula (1A-4) and the group represented by the hydrogen atom as R¹¹¹ corresponds to a sulfo group. In addition, a group in which a hydrogen atom is dissociated as a dissociative hydrogen atom from this carboxy group corresponds to a carboxy group having an ionic structure or a salt structure, and a group in which a hydrogen atom is dissociated as a dissociative hydrogen atom from this sulfo group corresponds to a sulfo group having an ionic structure or a salt structure.

Further, L³ may be a linking group obtained by combining each group represented by any one of Formula (1-1) to (1-8) or a linking group obtained by combining these groups, with at least one or more of an alkylene group, an alkenylene group, an alkynylene group, and an arylene group, or it may be a linking group obtained by linking two or more kinds of groups represented by any of Formula (1-1) to (1-8) or two or more linking group obtained by combining these groups, through a group obtained by combining one or two or more of an alkylene group, an alkenylene group, an alkynylene group, an arylene group, and a heteroarylene group.

Specific examples of the linking group obtained by combining two or more kinds of groups that can be adopted as L³ include a group obtained by combining at least 2 kinds (preferably 2 to 4 kinds) of alkylene groups, alkenylene groups, alkynylene groups, arylene groups, and heteroarylene groups; and a linking group obtained by combining at least one kind (preferably 1 to 4 kinds) of an alkylene group, an arylene group, or an heteroarylene group with at least one kind (preferably 1 to 4 kinds) of a group represented by any of Formula (1-1) to (1-8).

(R¹¹¹)

In a case where the hydrogen atom that can be adopted as R¹¹¹ is a dissociative hydrogen atom, the dissociative hydrogen atom may be dissociated from the group represented by Formula (A) to form an ionic structure or a salt structure. This is the same in the following description pertaining to R¹¹¹.

The description that a hydrogen atom is dissociative means, for example, that the acid dissociation constant (pKa) is 10 or less, preferably 7 or less, and more preferably 5 or less. The acid dissociation constant means a value at 25° C. in water.

The alkyl group, the alkenyl group, the alkynyl group, the aryl group, the heteroaryl group, and the monovalent aliphatic heterocyclic group, which can be adopted as R¹¹¹, are respectively synonymous with the corresponding groups in the substituent group T, and the same applies to the preferred ones.

All the alkyl group, the alkenyl group, the alkynyl group, the aryl group, the heteroaryl group, and the monovalent aliphatic heterocyclic group, which can be adopted as R¹¹¹, may be an unsubstituted group or a group having a substituent.

The substituent which may be contained in each of the above groups that can be adopted as R¹¹¹ is not particularly limited, and examples thereof include a group selected from a substituent group T described later, where a halogen atom is preferable. Examples of the halogen atom include a fluorine atom, a chlorine atom, a bromine atom, and an iodine atom, where a fluorine atom is preferable. In addition, a substituent capable of being bonded to a biological substance described later may be contained.

The number of substituents which may be contained in each of the above groups that can be adopted as R¹¹¹ is not particularly limited as long as it can be adopted for the structure, and it may be at least one. The upper limit value thereof is not particularly limited, and for example, all hydrogen atoms in the alkyl group, the alkenyl group, the alkynyl group, the heteroaryl group, and the monovalent aliphatic heterocyclic group may be substituted with a substituent.

Among the alkyl groups that can be adopted as R¹¹¹, preferred examples of the alkyl group having a substituent include a halogenoalkyl group. The halogenoalkyl group that can be adopted as R¹¹¹ is synonymous with the alkyl group in the substituent group T, except that at least one hydrogen atom in the alkyl group in the substituent group T is substituted with a halogen atom, and the same applies to the preferred one thereof.

Examples of the halogen atom include a fluorine atom, a chlorine atom, a bromine atom, and an iodine atom, where a fluorine atom is preferable.

The number of halogen atoms that constitute the halogenoalkyl group is not particularly limited, and the halogenoalkyl group may be, for example, a perhalogenoalkyl group.

Specifically, examples of the preferred form of the group represented by Formula (A) include the following groups (1) and (2). However, in these examples, the form in which the substituents in L³ and R¹¹¹ have a substituent is not excluded, and it may be unsubstituted or may have a substituent. Regarding the substituent which may be contained in the substituents in L³ and R¹¹¹, the descriptions for the above-described substituent which may be contained in L³ and R¹¹¹ can be applied, respectively.

(1) In a Case of Having a Group Represented by any One of General Formula (1-1) to (1-8)

In the group represented by Formula (A), L³ is a single bond or a linking group obtained by combining one or two or more kinds among an alkylene group, an alkenylene group, an alkynylene group, an arylene group, a heteroarylene group, and each group represented by any one of Formula (1-1) to Formula (1-8), and R¹¹¹ is a hydrogen atom, an alkyl group, an alkenyl group, an alkynyl group, an aryl group, a heteroaryl group, or a monovalent aliphatic heterocyclic group.

However, at least one among the groups represented by any of Formula (1-1) to Formula (1-8) is contained. This is the same in the following description pertaining to (1).

In this case, L³ is preferably a single bond or a linking group obtained by combining one or two or more kinds among an alkylene group, an alkenylene group, an alkynylene group, an arylene group, a heteroarylene group, and each group represented by any one of represented by Formula (1-1), Formula (1-3), Formula (1-4), or Formula (1-7), more preferably a single bond or a linking group obtained by combining one or two or more kinds among an alkylene group, an arylene group, a heteroarylene group, and each group represented by Formula (1-1), Formula (1-3), Formula (1-4), or Formula (1-7), still more preferably a linking group obtained by combining one or two or more kinds among an alkylene group, an arylene group, a heteroarylene group, and each group respectively represented by Formula (1-1), Formula (1-3), Formula (1-4), or Formula (1-7), and particularly preferably a linking group obtained by combining one or two or more kinds among an alkylene group, an arylene group, a heteroarylene group, and each group represented by Formula (1-1), Formula (1-3), or Formula (1-4). In these cases, R¹¹¹ is preferably a hydrogen atom, an alkyl group, an aryl group, a heteroaryl group, or a monovalent aliphatic heterocyclic group, more preferably a hydrogen atom or an alkyl group, and still more preferably a hydrogen atom.

(2) In a Case of not Having a Group Represented by any One of General Formula (1-1) to (1-8)

In the group represented by Formula (A), L³ is a single bond or a linking group obtained by combining one or two or more kinds among an alkylene group, an alkenylene group, an alkynylene group, an arylene group, and a heteroarylene group, and R¹¹¹ is a hydrogen atom, an alkyl group, an alkenyl group, an alkynyl group, an aryl group, a heteroaryl group, or a monovalent aliphatic heterocyclic group.

In this case, It is preferable that L³ is a single bond and R¹¹¹ is a hydrogen atom, an alkyl group, an alkenyl group, an aryl group, or a heteroaryl group, or L³ is an alkenylene group or an alkynylene group and R¹¹¹ is an alkyl group or an aryl group, and it is more preferable that L³ is a single bond and R¹¹¹ is a hydrogen atom, an alkyl group, an alkenyl group, or an aryl group.

1) R¹ to R⁶

R¹ to R⁶ represent a halogen atom, a cyano group, or a group represented by Formula (A), are preferably a halogen atom or a group represented by Formula (A), are more preferably a hydrogen atom, a halogen atom, or an alkyl group, or a sulfo group, and are still more preferably a hydrogen atom or an alkyl group.

Examples of the halogen atom that R¹ to R⁶ can adopt include a fluorine atom, a chlorine atom, a bromine atom, and an iodine atom, where a chlorine atom is preferable.

As the alkyl group that can be adopted as R¹ to R⁶, the description of the alkyl group that can be adopted as R¹¹¹ can be applied.

Preferred examples of the substituent which may be contained in the alkyl group that can be adopted by R¹ to R⁶ include a sulfo group.

Specific examples of R¹ to R⁶ include a hydrogen atom, a methyl group, an ethyl group, a chlorine atom, a sulfo group, and a sulfomethyl group.

2) Z

Z represents the group represented by Formula (A) and is preferably a group having a preferred form according to the above (1) or (2), where it is preferably an alkyl group, an alkoxy group, an amino group, an aryl group, or a heteroaryl group, more preferably an alkyl group, an aryl group, or a heteroaryl group, and still more preferably an aryl group.

As the alkyl group, the alkoxy group, the amino group, the aryl group, and the heteroaryl group, which can be adopted by Z, the description of the alkyl group, the alkoxy group, the amino group, the aryl group, and the heteroaryl group, as the group represented by Formula (A), can be applied.

Z preferably has a carboxy group or a substituent capable of being bonded to a biological substance described later.

In the description pertaining to each group that can be adopted as Z below, a form that has, instead of the carboxy group, a substituent capable of being bonded to a biological substance described later, is also preferable.

The alkyl group, the alkoxy group, and the amino group, which can be adopted by Z, preferably have a carboxy group, where —(CH₂)ₙCOOH, —O(CH₂)COOH, or —NH(CH₂)ₙCOOH is more preferable. It is noted that n is an integer of 1 to 15, preferably an integer of 1 to 10, and more preferably an integer of 1 to 5.

The heteroaryl group that can be adopted by Z preferably has a carboxy group.

The heteroaryl group is preferably a monocyclic group, more preferably a thiophene ring group or an imidazole ring group, and still more preferably an imidazole ring group.

The aryl group that can be adopted by Z is preferably a phenyl group and more preferably a group represented by Formula (C).

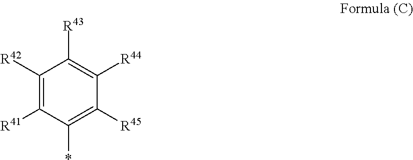

In the formula, R⁴¹ to R⁴⁵ represent a hydrogen atom or a substituent. * represents a bonding portion.

However, at least one of (C-1), (C-2), or (C-3) is satisfied.

(C-1) at least one of R⁴², R⁴³, or R⁴⁴ is a group having any of a carboxy group or a substituent capable of being bonded to a biological substance.

(C-2) R⁴¹ and R⁴⁵ are an alkyl group or a halogen atom.

(C-3) at least one of R⁴¹ or R⁴⁵ is a fluorine atom or an alkoxy group.

In a case where the above (C-1) is satisfied, nonradiative deactivation is suppressed, in a case where the above (C-2) is satisfied, heat deactivation due to the free rotation of the aryl group is suppressed by the steric hindrance, in a case where the above (C-3) is satisfied, heat deactivation due to the free rotation of the aryl group is suppressed by the intramolecular hydrogen bond, and in a case where at least one of these (C-1) to (C-3) are satisfied, the fluorescence quantum yield is further improved.

It is noted that in a case where both R⁴¹ and R⁴⁵ are a fluorine atom, it is determined that both of the above (C-2) and (C-3) are satisfied.

Examples of the substituent that can be adopted by R⁴¹ to R⁴⁵ include a hydrogen atom, a halogen atom, a cyano group, an alkyl group, an alkoxy group, a carboxy group, an amide group, a sulfo group, a sulfoamide group, an amino group, an alkenyl group, an aryl group, a heteroaryl group, an aliphatic heterocyclic group, or a substituent capable of being bonded to a biological substance described later, where a halogen atom, an alkyl group, an alkoxy group, a carboxy group, an amide group, or a substituent capable of being bonded to a biological substance described later is preferable.

The alkyl group, the alkoxy group, the amide group, the sulfoamide group, the amino group, the alkenyl group, the aryl group, the heteroaryl group, or the aliphatic heterocyclic group, which can be adopted as R⁴¹ to R⁴⁵, may have a substituent, examples of which include a group in the substituent group T described later. Preferred examples thereof include a poly(alkyleneoxy) group, a sulfo group, a carboxy group, or a substituent capable of being bonded to a biological substance.

From the viewpoint of suppressing the rotation of the benzene ring in Formula (C), the substituent in R⁴¹ and R⁴⁵ is preferably a halogen atom, an alkyl group, or an alkoxy group.

The substituent that can be adopted as R⁴² to R⁴⁴ is not particularly limited, and it is preferably selected from the substituent group T described later and preferably has a carboxy group or a substituent capable of being bonded to a biological substance. The carboxy group or the substituent capable of being bonded to a biological substance may be directly bonded to the benzene ring in General Formula (C) or may be bonded through a linking group; however, it is preferably directly bonded. Preferred examples of the linking group include an alkylene group, an alkyleneoxy group, an amide group, or a group formed by combining these groups. Examples of the alkylene group, the alkyleneoxy group, the amide group, or a group obtained by combining these groups include —CONH(CH₂CH₂O)ₘCH₂CH₂—. m is an integer of 1 to 30, preferably an integer of 1 to 15, and more preferably an integer of 1 to 10.

Among them, it is preferable that R⁴³ has a carboxy group or a substituent capable of being bonded to a biological substance from the viewpoint of the binding property to a biological substance.

R⁴² to R⁴⁴ are preferably a hydrogen atom, or a carboxy group or a group having a substituent capable of being bonded to a biological substance, and more preferably a hydrogen atom, a carboxy group, or a substituent capable of being bonded to a biological substance.

As each of the halogen atom, the alkyl group, and the alkoxy group, which can be adopted as R⁴¹ to R⁴⁵, the corresponding group in the substituent group T can be applied.

3) R⁷ to R¹²

The fluorescent compound according to the embodiment of the present invention has ═N⁺R⁷R⁸ or ═NR⁹ at the 3-position and has a substituent Y at the 6-position, in a ring structure in which the carbon atom at the 10-position of the anthracene ring is replaced with a silicon atom or a phosphorus atom as represented by any one of General Formula (I) to (IV). The substituent Y is —NR¹⁰R¹¹ or —OR¹².

R⁷ to R¹² represent the group represented by Formula (A), and the preferred form according to the above (1) or (2) can be preferably applied thereto.

Here, R⁷ and R⁸ may be bonded to each other to form a 4- to 7-membered aliphatic heterocyclic ring, and R¹⁰ and R¹¹ may be bonded to each other to form a 4- to 7-membered aliphatic heterocyclic ring. In addition, R⁷ to R¹¹ each may be bonded to adjacent R² to R⁵ to form a 5- to 7-membered aliphatic heterocyclic ring or a 5- to 7-membered aromatic heterocyclic ring.

All of the ring which may be formed by bonding R⁷ and R⁸ to each other, the ring which may be formed by bonding R¹⁰ and R¹¹ to each other, or the ring which may be formed by bonding each of R⁷ to R^{u} to adjacent R² to R⁵, which are described above, may have, in addition to the nitrogen atom to which R⁷ to R¹¹ are bonded, 1 to 3 heteroatoms selected from an oxygen atom, a nitrogen atom, and a sulfur atom, as the ring-constituting atom, where a sulfur atom is preferable.

R⁷ and R⁸ are preferably a hydrogen atom, an alkyl group, or an aryl group, and more preferably a hydrogen atom or an alkyl group.

As the alkyl group or the aryl group, which can be adopted by R⁷ and R⁸, the description of the alkyl group or the aryl group, as the group represented by Formula (A), can be applied.

Preferred examples of the substituent which may be contained in the alkyl group or the aryl group, which can be adopted by R⁷ and R⁸, include electron withdrawing groups described later, such as a halogen atom and a carbonyl group. Among them, R⁷ and R⁸ are preferably an alkyl group and more preferably an alkyl group substituted with a fluorine atom.

In a case where R⁷ and R⁸ are not bonded to any of the groups, R⁷ and R⁸ may be the same or may be different from each other; however, they are preferably the same.

The 4- to 7-membered aliphatic heterocyclic ring that can be formed by bonding R⁷ and R⁸ to each other is not particularly limited; however, it is preferably a saturated aliphatic heterocyclic ring, which preferably has 4 to 6 ring members. This aliphatic heterocyclic ring may be a monocyclic ring or may have a fused ring structure such as a structure in which at least one atom in the norbornane structure is replaced with a nitrogen atom. In addition, it is also preferable that the ring-constituting atom has >SO₂, and it is also preferable that the substituent has an electron withdrawing group described later, such as a halogen atom (preferably a fluorine atom).

Both the ring that can be formed by bonding R⁷ to R⁵ and the ring that can be formed by bonding R⁸ to R⁴ are may be aliphatic or may be aromatic as long as they are a heterocyclic ring having 5 to 7 ring members. In these rings, the number of ring members is preferably 5 or 6, and it is preferable that a heteroatom other than the nitrogen atom at which R⁷ or R⁸ is substituted is not contained as the ring-constituting atom. In addition, a monocyclic ring or a fused ring may be good; however, the ring formed by the bonding is preferably a monocyclic ring.

As R¹⁰ and R¹¹, the description in R⁷ and R⁸ can be applied. However, there is a difference is that the nitrogen atom to which R⁷ and R⁸ are bonded has a positive charge, whereas the nitrogen atom to which R¹⁰ and R¹¹ are bonded does not have a positive charge. In addition, the ring that can be formed by bonding R⁷ to R⁵ is read as a ring that can be formed by bonding R¹⁰ to R², or as a ring that can be formed by bonding R¹¹¹ to R³.

R⁹ is preferably an alkyl group.

As the alkyl group that can be adopted as R⁹, the description of the alkyl group that can be adopted as R⁷ and R⁸ can be applied. The ring that can be formed by bonding R⁷ to R⁵ is read as a ring that can be formed by bonding R⁹ to adjacent R⁴ or R⁵.

R¹² is preferably a hydroxy group, an acyl group, or a sulfonyl group.

As the acyl group or the sulfonyl group, which can be adopted by R¹², the description of the alkyl group that can be adopted by R⁷ and R⁸ can be applied. The ring that can be formed by bonding R⁷ to R⁵ is read as a ring that can be formed by bonding R⁹ to adjacent R⁴ or R⁵.

4) Y

Y represents —NR¹⁰R¹¹ or —OR¹² and is preferably —NR¹⁰R¹¹.

R¹⁰ to R¹² are as described in R¹⁰ to R¹².

5) R²¹ and R²²

R²¹ and R²² represent the group represented by Formula (A), and the preferred form according to the above (1) or (2) can be preferably applied thereto.

R²¹ and R²² may be bonded to each other to form a 4- to 7-membered aliphatic heterocyclic ring,

However, at least one of R²¹ or R²² is an alkenyl group, an alkynyl group, an aryl group, or a heteroaryl group.

In addition, in a case where R²¹ is an aryl group and R²² is an alkyl group or an aryl group, in at least one of a combination of R⁷ and R⁸ or a combination of R¹⁰ and R¹¹, R⁷ and R⁸, or R¹⁰ and R¹¹ are bonded to each other to form a 4-membered aliphatic heterocyclic ring. That is, in a case where the fluorescent compound according to the embodiment of the present invention has R¹⁰ and R¹¹, in at least one of the combination of R⁷ and R⁸ or the combination of R¹⁰ and R¹¹, R⁷ and R⁸, or R¹⁰ and R¹¹ are bonded to each other to form a 4-membered aliphatic heterocyclic ring, and in a case where the fluorescent compound according to the embodiment of the present invention does not have R¹⁰ and R¹¹, R⁷ and R⁸ are bonded to each other to form a 4-membered aliphatic heterocyclic ring.

The description pertaining to these R²¹ and R²² is the same as the following description pertaining to R²¹ and R²².

In a case where at least one of R²¹ or R²² has an alkenyl group, an alkynyl group, an aryl group, or a heteroaryl group, it is conceived that these substituents have large steric bulkiness and improve the stability of the silicon atom by suppressing the approach of a photolysis causing substance such as active oxygen to the silicon atom, whereby excellent light resistance can be exhibited. In a case where R²¹ is an aryl group and R²² is an alkyl group or an aryl group, it is presumed that the fluorescence quantum yield is decreased by the nonradiative deactivation due to the free rotation of the aryl group, and thus in a case where in at least one of the combination of R⁷ and R⁸ or the combination of R¹⁰ and R¹¹, R⁷ and R⁸, or R¹⁰ and R¹¹ are further bonded to each other to form a 4- to 7-membered aliphatic heterocyclic ring, it is conceived that the fluorescence quantum yield is improved by suppressing TICT described later, and high brightness can be maintained.

R²¹ and R²² are preferably an alkyl group, an alkenyl group, an alkynyl group, an aryl group, or a heteroaryl group.

As the alkyl group, the alkenyl group, the alkynyl group, the aryl group, or the heteroaryl group, which can be adopted by R²¹ and R²², the description of the alkyl group, the alkenyl group, the alkynyl group, the aryl group, or the heteroaryl group, as the group represented by Formula (A), can be applied.

Preferred examples of the substituent which may be contained in the alkyl group, the alkenyl group, the alkynyl group, the aryl group, or the heteroaryl group, which can be adopted by R²¹ and R²², include a carboxy group, an alkoxy group, and an aryl group. Among these, preferably examples thereof include an alkenyl group substituted with a carboxy group or an alkoxy group, and an alkenyl group or alkynyl group substituted with an aryl group.

Preferred examples of the combination of R²¹ and R²² include a combination of an alkyl group with an alkenyl group, an alkynyl group, an aryl group, or a heteroaryl group, a combination of an alkenyl group with an alkenyl group, a combination of an alkynyl group with an alkynyl group, and a combination of a heteroaryl group with a heteroaryl group, where a combination of an alkyl group with an alkenyl group or an aryl group is more preferable.

6) R²³

R²³ represents a group represented by Formula (A) and is preferably a hydroxy group, an alkoxy group, an aryl group, or an alkyl group.

In a case where R²³ is a hydroxy group, the hydrogen atom in this hydroxy group may be dissociated to have an ionic structure or a salt structure. The same applies in the following hydroxy group that can be adopted as R²³.

(Fluorescent Compound Represented by Formula (I) or (II) or Salt Thereof)

Among the fluorescent compounds according to the embodiment of the present invention, the fluorescent compound represented by Formula (I) or (II) or a salt thereof (hereinafter, referred to as a “fluorescent compound represented by Formula (I) or (II)”) is a compound having a ring structure in which the carbon atom at the 10-position of the anthracene ring is replaced with a silicon atom, and it satisfies the following condition α in addition to the above-described regulation pertaining to each substituent.

(Condition α)

In a case where Z is an aryl group, this aryl group is a group represented by Formula (C).

In the fluorescent compound represented by Formula (I) or (II), in a case where Z is an aryl group, the fluorescence quantum yield is decreased by the heat deactivation or the like due to the free rotation of the aryl group, and thus it is not possible to maintain an excellent fluorescence quantum yield, unless this aryl group is the group represented by Formula (C), which satisfies at least one of the above (C-1), . . . , or (C-3).

The fluorescent compound represented by Formula (I) or (II) preferably includes the following compound.

R⁷, R⁸, R¹⁰, and R¹¹ are preferably a hydrogen atom, an alkyl group, or an aryl group. It is more preferable that they a hydrogen atom or an alkyl group having 1 to 3 carbon atoms or R⁷ and R¹, or R¹⁰ and R¹¹ are bonded to each other to form a 4- or 5-membered aliphatic heterocyclic ring.

Among the above, it is preferable that R⁷, R⁸, R¹⁰ and R¹¹ are a hydrogen atom or a methyl group or that R⁷ and R⁸, or R¹⁰ and R¹¹ are bonded to each other to form a 4-membered ring, where this 4-membered ring is an unsubstituted azetidine ring or an azetidine ring having a fluorine atom as a substituent, from the viewpoint that the nonradiative deactivation due to expansion and contraction of various molecular bonds in the substituent is suppressed to a minimum, and as a result, a high fluorescence quantum yield can be exhibited.

In addition, it is preferable that in at least one of the combination of R⁷ and R⁸ or the combination of R¹⁰ and R¹¹, R⁷ and R⁸, or R¹⁰ and R¹¹ are bonded to each other to form a 4- to 7-membered aliphatic heterocyclic ring, from the viewpoint that the fluorescence quantum yield is improved as a result of the suppression of TICT. However, in a case where R²¹ is an aryl group and R²² is an alkyl group or an aryl group, the ring formed by bonding R⁷ and R⁸, or R¹⁰ and R¹¹ to each other in at least one of the combination of R⁷ and R⁸ or the combination of R¹⁰ and R¹¹ is a 4-membered aliphatic heterocyclic ring.

It is preferable that in a case where at least one of R⁷, . . . , or R¹¹ is a group including an electron withdrawing group, that is, in a case where the fluorescent compound according to the embodiment of the present invention has R¹⁰ and R¹¹, in the fluorescent compound represented by General Formula (I), at least one of R⁷, R⁸, R¹⁰ or R¹¹¹ is a group including an electron withdrawing group, and in the fluorescent compound represented by General Formula (II), at least one of R⁹, R¹⁰, or R¹¹ is a group including an electron withdrawing group, and in a case where the fluorescent compound according to the embodiment of the present invention does not have R¹⁰ and R¹¹, in the fluorescent compound represented by General Formula (I), at least one of R⁷ or R⁸ is a group including an electron withdrawing group, and in the fluorescent compound represented by General Formula (II), R⁹ is a group including an electron withdrawing group, from the viewpoint that the fluorescence quantum yield is improved as a result of the suppression of TICT.

The TICT will be described later, and it is presumed that due to having an electron withdrawing group, the ionization potential of the nitrogen atom of the amino group contained at the 3-position or the 6-position of the xanthene skeleton can be increased, whereby the TICT can be suppressed.

The electron withdrawing group refers to a group having a characteristic of increasing an ionization potential of an amino group contained at a 3-position or a 6-position by an inductive effect and/or a mesomeric effect. Examples of the electron withdrawing group include a halogen atom (a fluorine atom, a chlorine atom, a bromine atom, or an iodine atom), a nitro group, a cyano group, a sulfonyl group, a phosphoryl group, an azido group (—N₃), a carbonyl group, an alkoxycarbonyl group, an aryloxycarbonyl group, and a heterocyclic oxycarbonyl group, where a halogen atom or a sulfonyl group is preferable. Examples of the alkoxycarbonyl group, the aryloxycarbonyl group, and the heterocyclic oxycarbonyl group respectively include respective groups in the substituent group T described later. In addition, as the sulfonyl group, an alkyl, cycloalkyl, or arylsulfonyl group in the substituent group T described later can be referred to.

The number of electron withdrawing groups is not particularly limited as long as it is one or more, where one or two electron withdrawing groups are preferable.

As described above, it is preferable that Z is a group represented by Formula (C) from the viewpoint of improving a fluorescence quantum yield by suppressing the free rotation of the aryl group.

It is preferable that Y is —NR¹⁰R¹¹ from the viewpoint of wavelength compatibility with a general-purpose fluorescence microscope filter.

From the viewpoint of further improving the fluorescence quantum yield, the fluorescent compound represented by Formula (I) or (II) is preferably a compound represented by General Formula (IA) or (IIA) or a salt thereof.

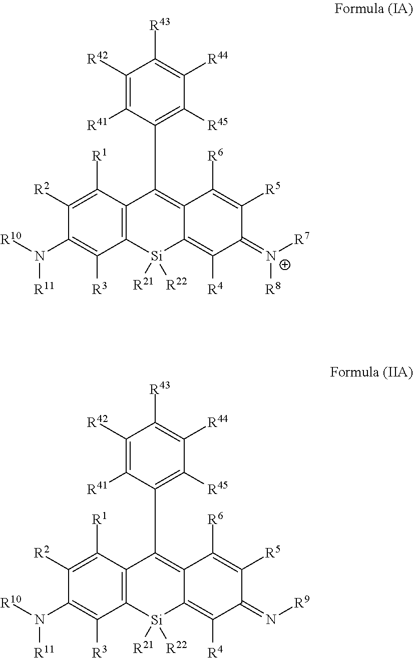

In the formulae, R¹ to R¹¹ and R⁴¹ to R⁴⁵ respectively have the same meanings as R¹ to R¹¹ and R⁴¹ to R⁴⁵.

R²¹ and R²² represent the group represented by Formula (A), and R²¹ and R²² may be bonded to each other to form a 4- to 7-membered aliphatic heterocyclic ring. However, at least one of R²¹ or R²² is an alkenyl group or an aryl group.

In addition, in a case where R²¹ is an aryl group and R²² is an alkyl group or an aryl group, in at least one of the combination of R⁷ and R⁸ or the combination of R¹⁰ and R¹¹, R⁷ and R¹, or R¹⁰ and R¹¹ are bonded to each other to form a 4-membered aliphatic heterocyclic ring. That is, in a case where the fluorescent compound according to the embodiment of the present invention has R¹⁰ and R¹¹, in at least one of the combination of R⁷ and R⁸ or the combination of R¹⁰ and R¹¹, R⁷ and R⁸, or R¹⁰ and R¹¹ are bonded to each other to form a 4-membered aliphatic heterocyclic ring, and in a case where the fluorescent compound according to the embodiment of the present invention does not have R¹⁰ and R¹¹, R⁷ and R⁸ are bonded to each other to form a 4-membered aliphatic heterocyclic ring.

One of the preferred embodiments of the present invention includes a fluorescent compound or a salt thereof, which is represented by General Formula (IA), where R⁷, R⁸, R¹⁰, and R¹¹ are a hydrogen atom or an alkyl group having 1 to 3 carbon atoms, and R⁴¹ to R⁴⁵ satisfy (C-1) and (C-2), satisfy (C-1) and (C-3), or satisfy all of (C-1) to (C-3) in the condition α.

It is more preferably a fluorescent compound or a salt thereof, where R⁷, R⁸, R¹⁰, and R¹¹ are an alkyl group having 1 to 3 carbon atoms, and R⁴¹ to R⁴⁵ satisfy (C-1) and (C-3) or satisfy all of (C-1) to (C-3) in the condition α.

One of other preferred embodiments of the present invention includes a fluorescent compound or a salt thereof, which is represented by General Formula (IA), where in at least one of the combination of R⁷ and R⁸ or the combination of R¹⁰ and R¹¹, R⁷ and R⁸, or R¹⁰ and R¹¹ are bonded to each other to form a 4- to 7-membered aliphatic heterocyclic ring, and R⁴¹ to R⁴⁵ satisfy (C-1) and (C-2), satisfy (C-1) and (C-3), or satisfy all of (C-1) to (C-3) in the condition α.

It is more preferably a fluorescent compound or a salt thereof, where in the combination of R⁷ and R⁸ and the combination of R¹⁰ and R¹¹, R⁷ and R⁸, and R¹⁰ and R¹¹ are bonded to each other to form a 4- to 7-membered aliphatic heterocyclic ring, and R⁴¹ to R⁴⁵ satisfy (C-1) and (C-3) or satisfy all of (C-1) to (C-3) in the condition α, and it is still more preferably a fluorescent compound or a salt thereof, where in the combination of R⁷ and R⁸ and the combination of R¹⁰ and R¹¹, R⁷ and R⁸, and R¹⁰ and R¹¹ are bonded to each other to form a 4- or 5-membered aliphatic heterocyclic ring, and R⁴¹ to R⁴⁵ satisfy (C-1) and (C-3) or satisfy all of (C-1) to (C-3) in the condition α, and both R⁴¹ and R⁴⁵ are an alkoxy group regarding (C-3).

(Fluorescent Compound Represented by Formula (III) or (IV) or Salt Thereof)

Among the fluorescent compounds according to the embodiment of the present invention, the fluorescent compound represented by Formula (III) or (IV) or a salt thereof (hereinafter, referred to as a “fluorescent compound represented by Formula (III) or (IV)”) is a P-rhodamine compound or a P-rhodol compound, which has a ring structure in which an oxygen atom which is a ring-constituting atom of a xanthene skeleton is replaced with a phosphorus, and it satisfies the following condition β in addition to the above-described regulation pertaining to each substituent.

(Condition β)

In at least one of a combination of R⁷ and R⁸ or a combination of R¹⁰ and R¹¹, R⁷ and R⁸, or R¹⁰ and R¹¹ are bonded to each other to form a 4-membered aliphatic heterocyclic ring.

That is, in a case where the fluorescent compound according to the embodiment of the present invention has R¹⁰ and R¹¹, in at least one of the combination of R⁷ and R⁸ or the combination of R¹⁰ and R¹¹, R⁷ and R⁸, or R¹⁰ and R¹¹ are bonded to each other to form a 4-membered aliphatic heterocyclic ring, and in a case where the fluorescent compound according to the embodiment of the present invention does not have R¹⁰ and R¹¹, R⁷ and R⁸ are bonded to each other to form a 4-membered aliphatic heterocyclic ring.

In the fluorescent compound represented by Formula (III) or (IV), in a case where the fluorescent compound represented by Formula (III) or (IV) does not satisfy the condition β, twisted intramolecular charge transfer (TICT) occurs, and thus a decrease in fluorescence quantum yield occurs.

The TICT means that R⁷ and R⁸, or R¹⁰ and R¹¹ of the amino group contained at the 3-position or 6-position of the xanthene skeleton are subjected to a transition to a charge-separated state (=twisted state) in which they are present non-planarly with respect to the xanthene skeleton plane. In the present invention, it is presumed that at least one of the amino groups contained at the 3-position or the 6-position of the xanthene skeleton is a 4-membered ring having a large strain, thereby the planar state (=non-twisted state) is stabilized and the transitioning to the TICT state is suppressed.

The fluorescent compound represented by Formula (III) or (IV) preferably includes the following compound.

It is preferable that in a case where at least one of R⁷, . . . , or R¹¹ is a group including an electron withdrawing group, that is, in a case where the fluorescent compound according to the embodiment of the present invention has R¹⁰ and R¹¹, in the fluorescent compound represented by General Formula (III), at least one of R⁷, R⁸, R¹⁰ or R¹¹¹ is a group including an electron withdrawing group, and in the fluorescent compound represented by General Formula (IV), at least one of R⁹, R¹⁰, or R¹¹ is a group including an electron withdrawing group, and in a case where the fluorescent compound according to the embodiment of the present invention does not have R¹⁰ and R¹¹, in the fluorescent compound represented by General Formula (III), at least one of R⁷ or R⁸ is a group including an electron withdrawing group, and in the fluorescent compound represented by General Formula (IV), R⁹ is a group including an electron withdrawing group, from the viewpoint that the fluorescence quantum yield is improved as a result of the suppression of TICT.

Regarding the electron withdrawing group, the description of the electron withdrawing group that is preferably contained in at least one of R⁷, . . . , or R¹¹¹, which is described in the preferred form of the fluorescent compound represented by Formula (I) or (II), can be applied.

It is preferable that R²³ is a hydroxy group, an alkoxy group, an aryl group, or an alkyl group from the viewpoint of wavelength compatibility with a generally used microscope filter.

However, in a case where R²³ is a hydroxy group, the hydrogen atom in this hydroxy group may be dissociated.

Specific examples of the fluorescent compound represented by any one of General Formulae (I) to (IV) according to the embodiment of the present invention will be shown below. However, the present invention is not limited to these compounds or salts thereof.

In a compound represented by Formula (1) or (2) or a salt thereof, X, Y, Z, R¹ to R⁶, and R⁷ to R⁹ are contained so that the regulation of any formula of General Formulae (I) to (IV) according to the embodiment of the present invention is satisfied. * represents a bonding site.

In the following exemplified compounds, Me represents a methyl group, Et represents an ethyl group, Ac represents an acetyl group, Tf represents a trifluoromethylsulfonyl group, and Ph represents a phenyl group.

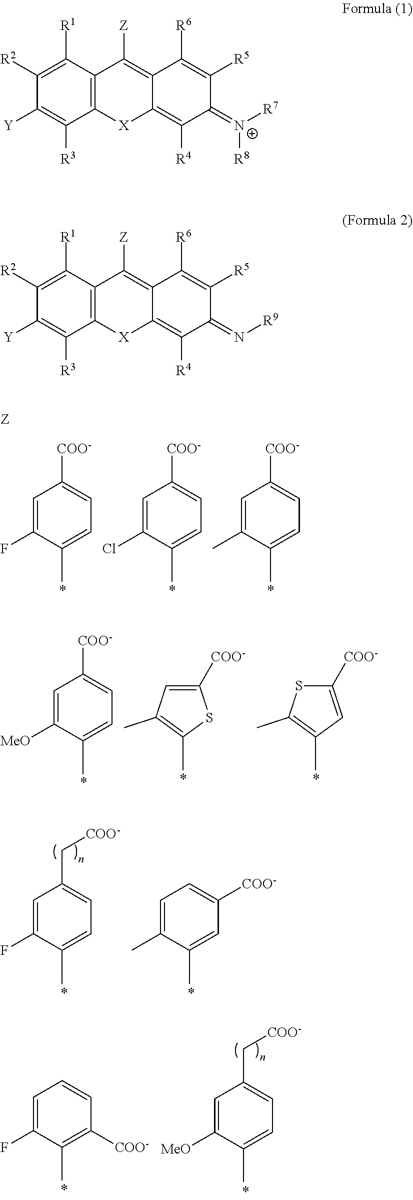

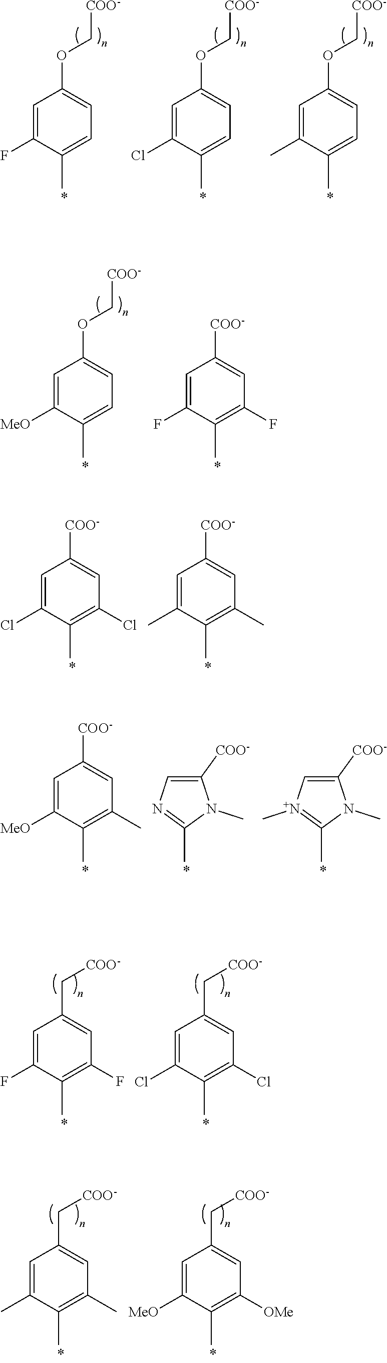

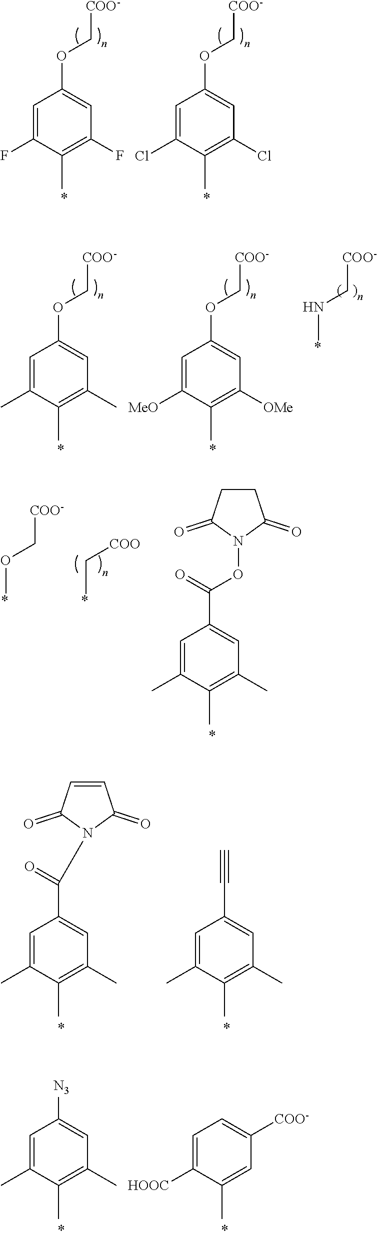

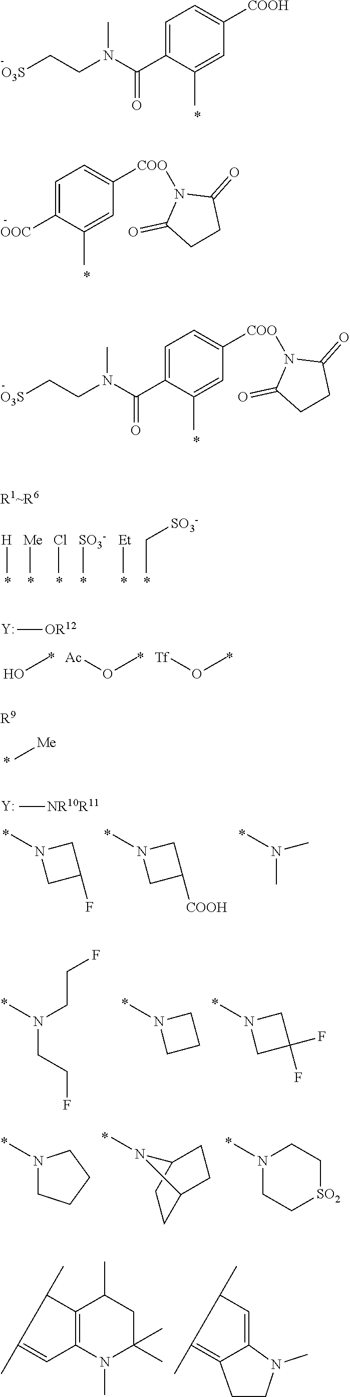

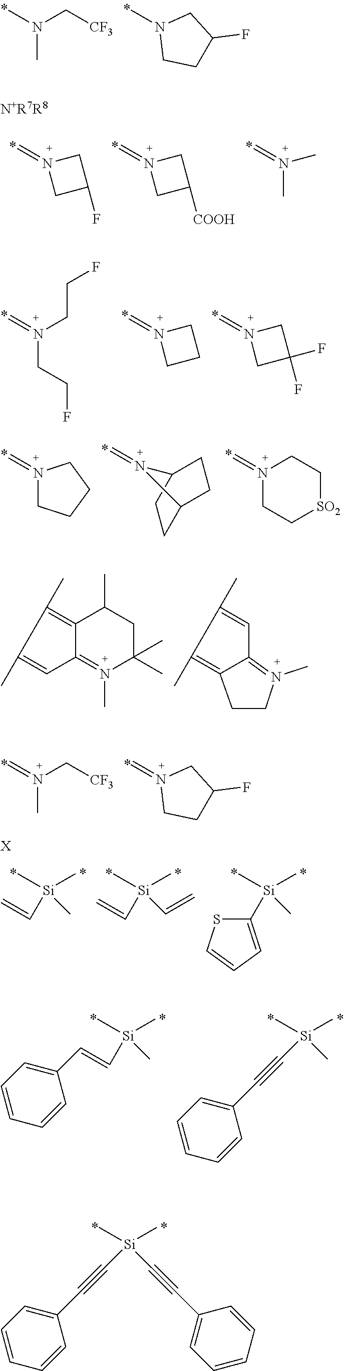

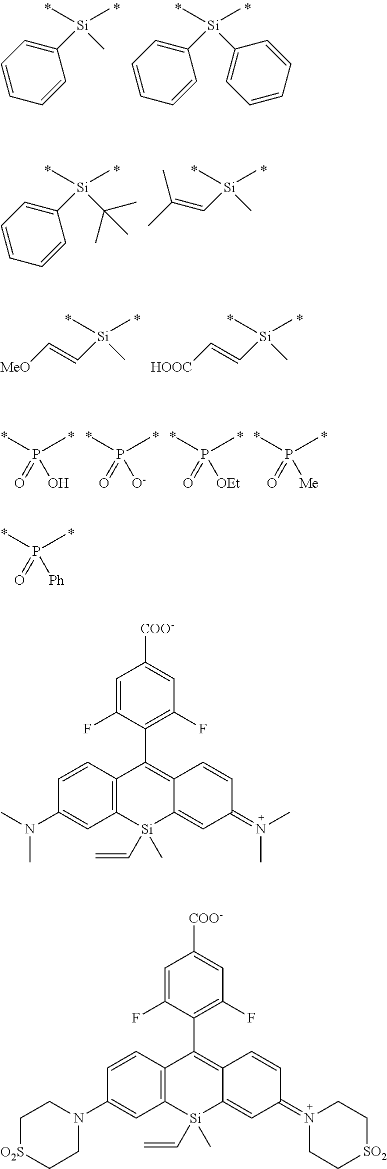

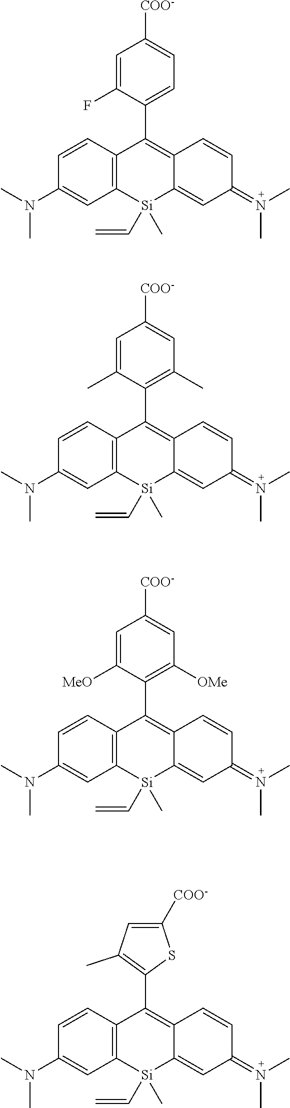

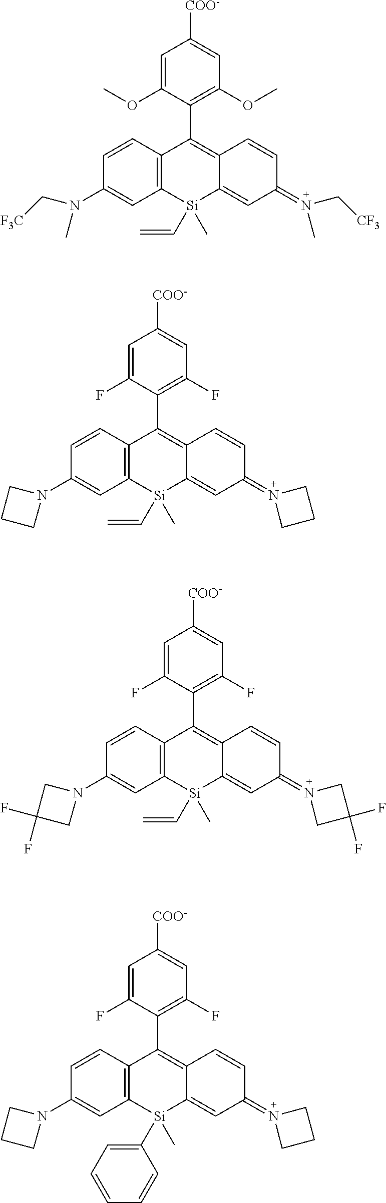

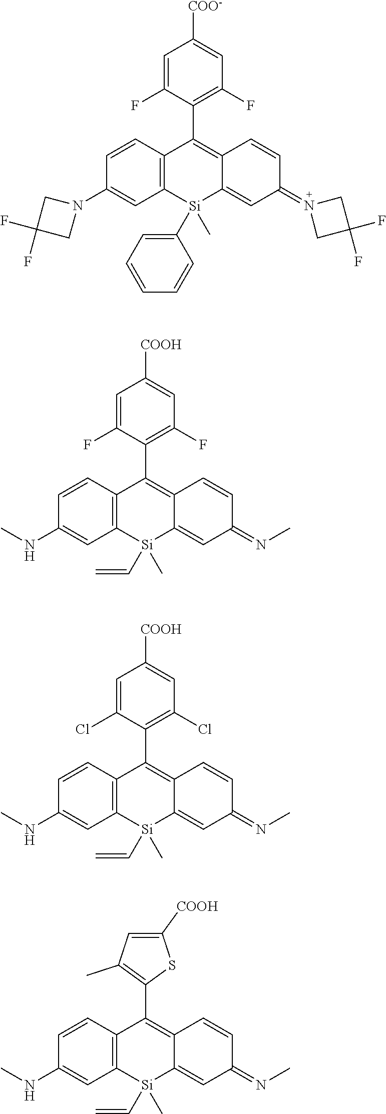

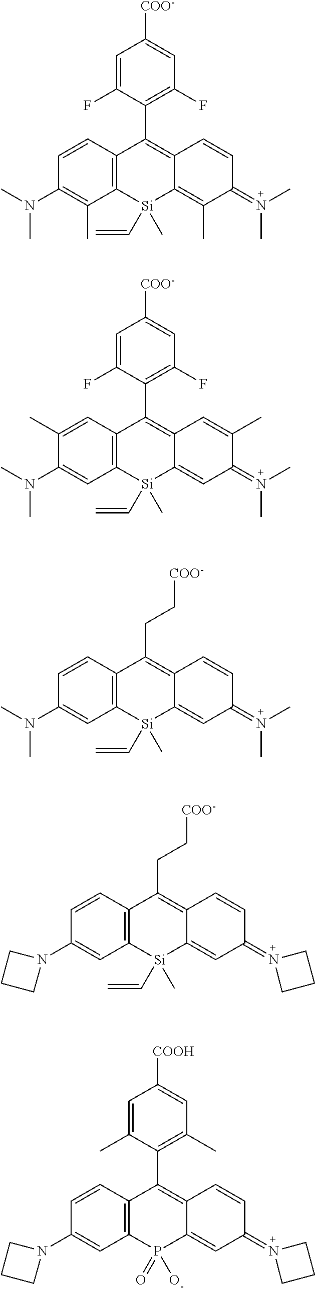

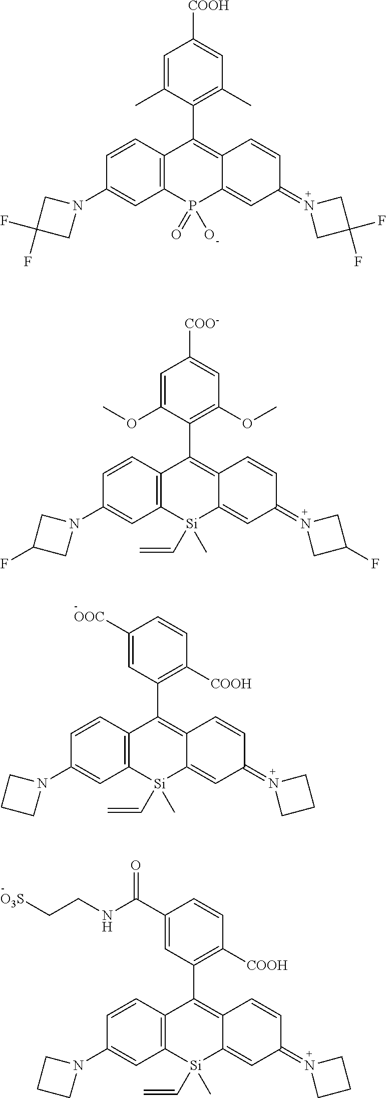

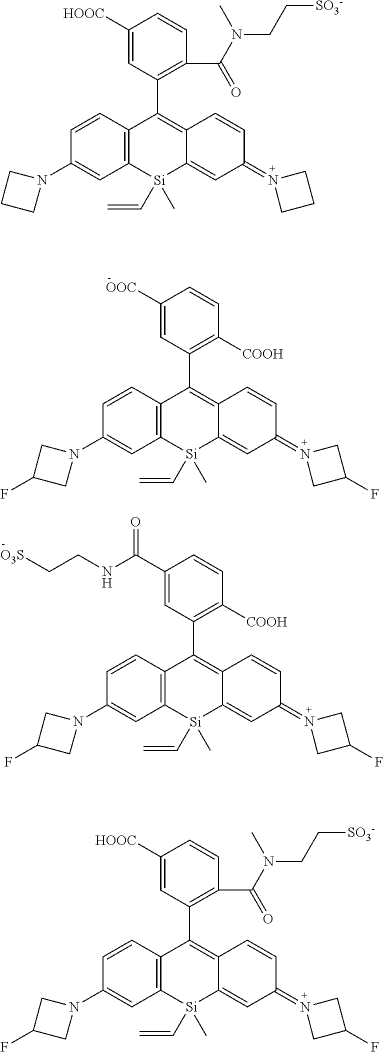

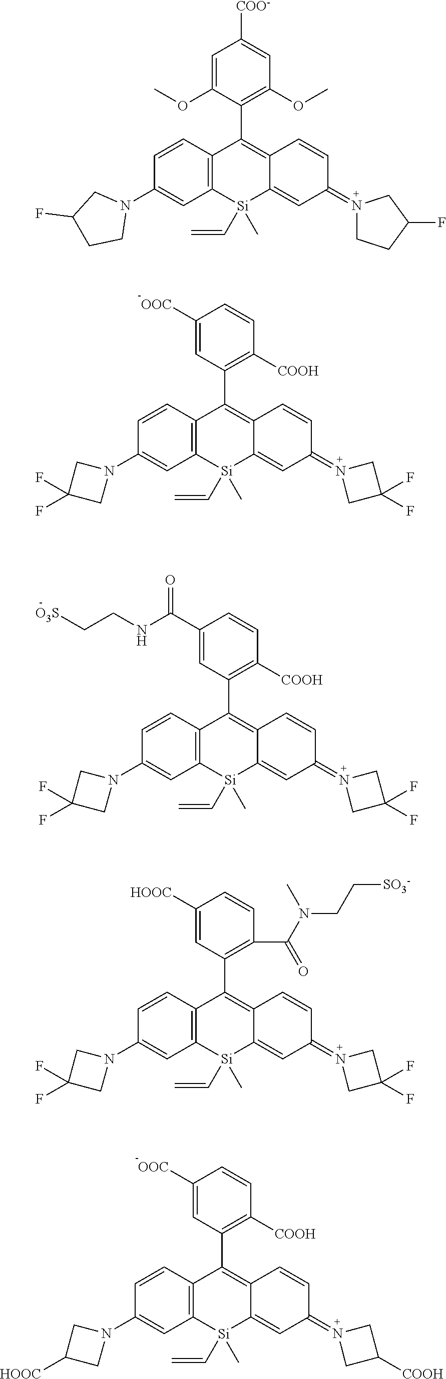

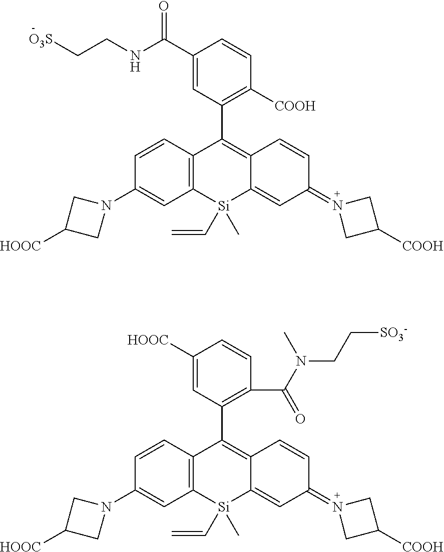

The fluorescent compound according to the embodiment of the present invention can be synthesized by a known method. For example, WO2020/033681A, WO2014/106957A, WO2014/144793A, US2018/0284105A, US2019/0100653A, U.S. Pat. No. 8,506,655B, and WO2018/043579A are mentioned.

The fluorescent compound according to the embodiment of the present invention has an excellent fluorescence quantum yield and light resistance and thus can be used as a reagent for in vivo fluorescence imaging by being bonded to a biological substance such as a protein, an amino acid, a nucleic acid, a sugar chain, and a lipid.

That is, the fluorescent compound according to the embodiment of the present invention includes a compound having a group capable of interacting (for example, physical adsorption and chemical bond) with a biological substance, and such a form is particularly preferable from the viewpoint of applying the fluorescent compound according to the embodiment of the present invention to the fluorescence labeling of the biological substance, such as in vivo fluorescence imaging.

The fluorescent compound according to the embodiment of the present invention preferably has at least one substituent capable of being bonded to a biological substance. The substituent having a substituent capable of being bonded to a biological substance is not particularly limited, and preferred examples thereof include Z and R¹¹ to R²³, where Z is more preferable.

Examples of the substituent capable of being bonded to a biological substance include a substituent capable of being bonded to a biological substance in the fluorescent compound according to the embodiment of the present invention, which is described in “the specific form in which the fluorescent compound according to the embodiment of the present invention and a biological substance interact with each other to be bonded to each other” described later, and preferred examples thereof include the following substituent. Among the fluorescent compounds according to the embodiment of the present invention, examples of the compound having a substituent capable of being bonded to a biological substance include a compound in which the above-described carboxy group in the fluorescent compound is replaced with a substituent capable of being bonded to a biological substance.

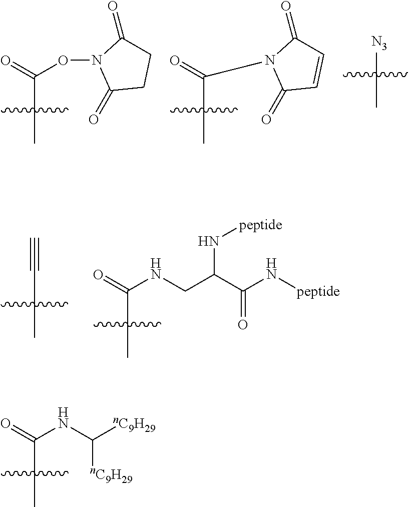

A compound having a group for acting (including adhesion) or bonding to a biological substance can be synthesized by a known method. For example, Bioconjugate Techniques (Third Edition, written by Greg T. Hermanson) can be referred to.

<<Fluorescently Labeled Biological Substance>>

The fluorescently labeled biological substance according to the embodiment of the present invention (also simply referred to as the labeled biological substance) is a substance in which the fluorescent compound according to the embodiment of the present invention is bonded to a biological substance. The bond between the fluorescent compound according to the embodiment of the present invention and a biological substance may have a form in which the fluorescent compound according to the embodiment of the present invention and the biological substance are directly bonded or a form of being linked via a linking group.

Preferred examples of the biological substance include a protein, an amino acid, a nucleic acid, a sugar chain, and a lipid. In the present invention, a protein is used to have a meaning including a peptide and refers to a compound in which two or more amino acids are bonded by peptide bonding. Preferred examples of the protein include an antibody, and preferred examples of the lipid include a phospholipid, a fatty acid, sterol. A labeled biological substance in which the fluorescent compound according to the embodiment of the present invention is bonded to an antibody is referred to as a labeled antibody.

Among the above biological substances, the clinically useful substance is not particularly limited, but examples thereof include immunoglobulins such as immunoglobulin (Ig) G, IgM, IgE, IgA, and IgD; blood plasma proteins such as complement, C-reactive protein (CRP), ferritin, α₁ microglobulin, β₂ microglobulin, and antibodies thereof, tumor markers such as α-fetoprotein, carcinoembryonic antigen (CEA), prostate acid phosphatase (PAP), carbohydrate antigen (CA) 19-9, and CA-125, and antibodies thereof, hormones such as luteinizing hormone (LH), follicle-stimulating hormone (FSH), human chorionic gonadotropin (hCG), estrogen, and insulin, and antibodies thereof, and viral infection-related substances of viruses such HIV and ATL, hepatitis B virus (HBV)-related antigens (HBs, HBe, and HBc), human immunodeficiency virus (HIV), adult T-cell leukemia (ATL), and antibodies thereof.

The examples thereof further include bacteria such as Corynebacterium diphtheria, Clostridium botulinum, mycoplasma, and Treponema pallidum, and antibodies thereof, protozoa such as Toxoplasma, Trichomonas, Leishmania, Trypanosoma, and malaria parasites, and antibodies thereof, embryonic stem (ES) cells such as ELM3, HMI, KH2, v6.5, v17.2, v26.2 (derived from mice, 129, 129/SV C57BL/6, and BALB/c), and antibodies thereof, antiepileptic drugs such as phenytoin and phenobarbital; cardiovascular drugs such as quinidine and digoxin; anti-asthma drugs such as theophylline; drugs such as antibiotics such as chloramphenicol and gentamicin, and antibodies thereof, and enzymes, extracellular toxins (for example, styrelidine O), and the like, and antibodies thereof. In addition, antibody fragments such as Fab′2, Fab, and Fv can also be used.

The specific form in which the fluorescent compound according to the embodiment of the present invention and a biological substance interact with each other to be bonded to each other includes, for example, the forms described below.
i) A non-covalent bond (for example, hydrogen bond, ionic bond including chelate formation) or a covalent bond between a peptide in the fluorescent compound according to the embodiment of the present invention and a peptide in the biological substance,
ii) Van der Waals force between a long-chain alkyl group in the fluorescent compound according to the embodiment of the present invention and a lipid bilayer, a lipid, or the like in the biological substance,
iii) an amide bond formed by reacting an N-hydroxysuccinimide ester (NHS ester) in the fluorescent compound according to the embodiment of the present invention with an amino group in the biological substance,
iv) a thioether bond formed by reacting a maleimide group in the fluorescent compound according to the embodiment of the present invention with a sulfanyl group (—SH) in the biological substance, and
v) a formation of a triazole ring, which is formed by a Click reaction between an azido group in the fluorescent compound according to the embodiment of the present invention and an acetylene group in the biological substance, or a Click reaction between an acetylene group in the fluorescent compound according to the embodiment of the present invention and an azido group in the biological substance.

In addition to the forms of the i) to v), the bond can be formed according to another form, for example, which is described in “Lucas C. D. de Rezende and Flavio da Silva Emery, A Review of the Synthetic Strategies for the Development of BODIPY Dyes for Conjugation with Proteins, Orbital: The Electronic Journal of Chemistry, 2013, Vol 5, No. 1, p. 62-83”. Further, the method described in the same document can be appropriately referred to for the preparation of the fluorescently labeled biological substance according to the embodiment of the present invention.

Among the fluorescent compounds according to the embodiment of the present invention, the labeled biological substance according to the embodiment of the present invention, which is obtained from a compound having a substituent capable of being bonded to a biological substance and a biological substance that is bonded to the compound by an interaction includes the compound in which a moiety other than the substituent capable of being bonded to a biological substance is replaced with a dye moiety in the fluorescent compound according to the embodiment of the present invention, and a product thereof, in the description of the compound example and the product in paragraph 0038 of JP2019-172826A. However, the present invention is not limited to these compounds or the like.

Since the fluorescently labeled biological substance according to the embodiment of the present invention uses the fluorescent compound according to the embodiment of the present invention exhibiting an excellent fluorescence quantum yield, it is possible to make it easier to identify a targeted biological substance. In addition, light resistance is excellent. In addition, since the fluorescently labeled biological substance according to the embodiment of the present invention uses the fluorescent compound according to the embodiment of the present invention exhibiting excellent light resistance, it can also be applied to an observation under more severe conditions such as a long-term observation or an observation using a strong laser as compared with a case of using a fluorescently labeled biological substance in the related art, which has Si— or P-rhodamine, or a rhodol compound. As a result, the fluorescently labeled biological substance according to the embodiment of the present invention can be applied to various use applications as in the observation of the biological substance.

<Reagent Containing Fluorescently Labeled Biological Substance>

In the reagent containing the fluorescently labeled biological substance according to the embodiment of the present invention, the form of the fluorescently labeled biological substance according to the embodiment of the present invention, for example, a solution form dissolved in an aqueous medium such as physiological saline and a phosphate buffer solution, and a solid form such as a fine particle powder or a lyophilized powder, is not particularly limited and can be appropriately selected depending on the purpose of use.

For example, in a case where the fluorescently labeled biological substance according to the embodiment of the present invention is used as a reagent for in vivo fluorescence imaging, it can be used as a reagent containing the fluorescently labeled biological substance having any one of the forms described above.

<Use Application of Fluorescently Labeled Biological Substance>

It is conceived that the fluorescently labeled biological substance according to the embodiment of the present invention, obtained from the fluorescent compound according to the embodiment of the present invention, makes it possible to stably detect fluorescence emitted from the fluorescent compound excited by light irradiation. Accordingly, the fluorescently labeled biological substance according to the embodiment of the present invention is suitable as, for example, a reagent for in vivo fluorescence imaging.

Cells stained by using the fluorescently labeled biological substance according to the embodiment of the present invention as a fluorescent dye can maintain the fluorescence intensity for a long time since the fading is highly inhibited. Accordingly, the fluorescently labeled biological substance according to the embodiment of the present invention can be suitably used in vivo fluorescence imaging which requires excellent light resistance, for example, a long-term observation of a biological substance such as the microscopic observation by time-lapse measurement, and an observation of a biological substance by using high-resolution microscopes such as the confocal laser microscope and the super-resolution microscope such as the stimulated emission depletion microscope (STED microscope).

In vivo fluorescence imaging using the fluorescently labeled biological substance according to the embodiment of the present invention includes the following processes of (i) to (iii).
(i) A process of preparing a targeted biological substance (hereinafter, referred to as “target biological substance”) and a fluorescently labeled biological substance according to the embodiment of the present invention in which a biological substance capable of binding to the target biological substance is bonded to the fluorescent compound according to the embodiment of the present invention
(ii) A process of bonding the target biological substance to the fluorescently labeled biological substance according to the embodiment of the present invention
(iii) A process of irradiating a substance obtained by bonding the fluorescently labeled biological substance according to the embodiment of the present invention to the target biological substance with light having a range of the wavelength which is absorbed by the fluorescently labeled biological substance according to the embodiment of the present invention, and detecting the fluorescence emitted by the fluorescently labeled biological substance according to the embodiment of the present invention

In the in vivo fluorescence imaging described above, examples of the biological substance capable of binding to the target biological substance include the above-described biological substance in the fluorescently labeled biological substance according to the embodiment of the present invention. The biological substance can be appropriately selected depending on the target biological substance (test object), and a biological substance capable of specifically binding to the test object can be selected.

Examples of the protein among the target biological substances include a protein, which is a so-called disease marker. The disease marker is not particularly limited, and examples thereof include α-fetoprotein (AFP), protein induced by vitamin K absence or antagonist II (PIVKA-II), breast carcinoma-associated antigen (BCA) 225, basic fetoprotein (BFP), carbohydrate antigen (CA) 15-3, CA19-9, CA72-4, CA125, CA130, CA602, CA54/61 (CA546), carcinoembryonic antigen (CEA), DUPAN-2, elastase 1, immunosuppressive acidic protein (IAP), NCC-ST-439, γ-seminoprotein (γ-Sm), prostate specific antigen (PSA), prostatic acid phosphatase (PAP), nerve specific enolase (NSE), Iba1, amyloid β, tau, squamous cell carcinoma associated antigen (SCC antigen), sialyl LeX-i antigen (SLX), SPan-1, tissue polypeptide antigen (TPA), sialyl Tn antigen (STN), cytokeratin (CYFRA) pepsinogen (PG), C-reactive protein (CRP), serum amyloid A protein (SAA), myoglobin, creatine kinase (CK), troponin T, and ventricular muscle myosin light chain I.

Examples of the bacterium among the above-described target biological substances include a bacterium to be subjected to a cellular and microbiological test, which are not particularly limited. Specific examples thereof include Escherichia coli, Salmonella, Legionella, and bacteria causing problems in public health.

The virus antigen among the above-described target biological substances is not particularly limited, and examples thereof include hepatitis virus antigens such as hepatitis C and B virus antigens, p24 protein antigen of HIV virus, and pp65 protein antigen of cytomegalovirus (CMV), and E6 and E7 proteins of papillomavirus (HPV).

In the above (i), the target biological substance is not particularly limited and can be prepared according to a conventional method.

In addition, the fluorescently labeled biological substance according to the embodiment of the present invention is not particularly limited and can be prepared by bonding a biological substance capable of binding to a target biological substance to the fluorescent compound according to the embodiment of the present invention, according to a conventional method. Examples of the form of the bond and the reaction for forming the bond include the form in which the bond is formed by interaction and the reaction which are described in <<fluorescently labeled biological substance>> according to the embodiment of the present invention.

In the above (ii), the fluorescently labeled biological substance according to the embodiment of the present invention may be directly bonded to the target biological substance or may be bonded through another biological substance which is different from the fluorescently labeled biological substance according to the embodiment of the present invention and the target biological substance. In vivo fluorescence imaging using the fluorescently labeled biological substance according to the embodiment of the present invention is not particularly limited, and examples thereof include fluorescent cell staining. The fluorescent cell staining includes a direct method in which a fluorescently labeled antibody is used as a primary antibody and an indirect method in which a primary antibody is reacted with a secondary antibody that is used as a fluorescently labeled antibody. The fluorescently labeled biological substance according to the embodiment of the present invention can be used as a fluorescently labeled antibody in both the direct method and the indirect method but is preferably used as a fluorescently labeled antibody in the indirect method.

The binding of the fluorescently labeled biological substance according to the embodiment of the present invention to the target biological substance is not particularly limited and can be carried out according to a conventional method.

In the above (iii), the wavelength for exciting the fluorescently labeled biological substance according to the embodiment of the present invention is not particularly limited as long as the wavelength is a wavelength (excitation wavelength) capable of exciting the fluorescently labeled biological substance according to the embodiment of the present invention. Generally, the wavelength for excitation is preferably 300 to 1,000 nm and more preferably 400 to 800 nm.

The fluorescence excitation light source used in the present invention is not particularly limited as long as it emits a wavelength (excitation wavelength) capable of exciting the fluorescently labeled biological substance according to the embodiment of the present invention, and various laser light sources can be used. Examples of the laser light source include a He—Ne laser, a CO₂ laser, an Ar ion laser, a Kr ion laser, a He—Cd laser, an excimer laser, a gas laser such as a nitrogen laser, a ruby laser, a yttrium-aluminum-garnet (YAG) laser, a solid-state laser such as glass laser, a dye laser, and a semiconductor laser. In addition, various optical filters can be used to obtain a preferred excitation wavelength or detect only fluorescence.

Other matters in the above (i) to (iii) are not particularly limited and can be appropriately selected depending on conditions such as a method, a reagent, and an apparatus, which are usually used.

In the in vivo fluorescence imaging using the fluorescently labeled biological substance according to the embodiment of the present invention, the fading of the substance obtained by bonding the fluorescently labeled biological substance according to the embodiment of the present invention to the target biological substance is highly inhibited, whereby a biological substance can be observed while maintaining the fluorescence intensity for a long time. Further, even in a case where a high resolution microscope or a super resolution microscope is used, observation can be carried out while maintaining the fluorescence intensity. In addition, since a substance obtained by bonding the fluorescently labeled biological substance according to the embodiment of the present invention to a target biological substance has high brightness, the substance can be easily identified.

Further, in addition to the above, the fluorescently labeled biological substance according to the embodiment of the present invention can be suitably used even in the long-term storage of stained cells, by appropriately adjusting the storage conditions.

—Substituent Group T—

In the present invention, the preferred substituents include those selected from the following substituent group T.

In addition, in the present specification, in a case where it is simply described as a substituent, the substituent refers to the substituent group T, and in a case where an individual group, for example, an alkyl group is only described, a corresponding group in the substituent group T is preferably applied.

Further, in the present specification, in a case where an alkyl group is described separately from a cyclic (cyclo)alkyl group, the alkyl group is used to include a linear alkyl group and a branched alkyl group. On the other hand, in a case where an alkyl group is not described separately from a cyclic alkyl group, and unless otherwise specified, the alkyl group is used to include a linear alkyl group, a branched alkyl group, and a cycloalkyl group. This also applies to groups (alkoxy group, alkylthio group, alkenyloxy group, and the like) containing a group capable of having a cyclic structure (alkyl group, alkenyl group, alkynyl group, and the like) and compounds containing a group capable of having a cyclic structure. In a case where a group is capable of forming a cyclic skeleton, the lower limit of the number of atoms of the group forming the cyclic skeleton is 3 or more and preferably 5 or more, regardless of the lower limit of the number of atoms specifically described below for the group that can adopt this structure,

In the following description of the substituent group T, a group having a linear or branched structure and a group having a cyclic structure, such as an alkyl group and a cycloalkyl group, are sometimes described separately for clarity.

As the groups included in the substituent group T, the following groups are included.

An alkyl group (preferably having 1 to 30 carbon atoms, more preferably 1 to 20 carbon atoms, still more preferably 1 to 12 carbon atoms, still more preferably 1 to 8 carbon atoms, still more preferably 1 to 6 carbon atoms, and particularly preferably 1 to 3 carbon atoms), an alkenyl group (preferably having 2 to 30 carbon atoms, more preferably 2 to 20 carbon atoms, still more preferably 2 to 12 carbon atoms, still more preferably 2 to 6 carbon atoms, and even still more preferably 2 to 4 carbon atoms), an alkynyl group (preferably having 2 to 30 carbon atoms, still more preferably 2 to 20 carbon atoms, still more preferably 2 to 12 carbon atoms, still more preferably 2 to 6 carbon atoms, and even still more preferably 2 to 4 carbon atoms), a cycloalkyl group (preferably having 3 to 20 carbon atoms), a cycloalkenyl group (preferably having 5 to 20 carbon atoms),

an aryl group (it may be a monocyclic group or may be a fused ring group (preferably a fused group in which 2 to 6 rings are fused); in a case of a fused ring group, it consists of a 5-membered to 7-membered ring; and the aryl group preferably has 6 to 40 carbon atoms, more preferably 6 to 30 carbon atoms, still more preferably 6 to 26 carbon atoms, and particularly preferably 6 to 10 carbon atoms),

a heterocycle group (it has, as a ring-constituting atom, at least one nitrogen atom, an oxygen atom, a sulfur atom, a phosphorus atom, a silicon atom, or selenium atom, may be a monocyclic ring, or may be a fused ring group (preferably a fused group in which 2 to 6 rings are fused); in a case of a monocyclic group, the monocyclic ring is preferably a 5-membered to 7-membered ring and more preferably a 5-membered or 6-membered ring; the heterocycle group preferably has 2 to 40 carbon atoms and more preferably 2 to 20 carbon atoms; and the heterocyclic group includes an aromatic heterocyclic group (a heteroaryl group) and an aliphatic heterocyclic group (an aliphatic heterocyclic group),

an alkoxy group (preferably having 1 to 20 carbon atoms, and more preferably having 1 to 12 carbon atoms), an alkenyloxy group (preferably having 2 to 20 carbon atoms, and more preferably having 2 to 12 carbon atoms), and an alkynyloxy group (preferably having 2 to 20 carbon atoms, and more preferably having 2 to 12 carbon atoms), a cycloalkyloxy group (preferably having 3 to 20 carbon atoms), an aryloxy group (preferably having 6 to 40 carbon atoms, more preferably having 6 to 26 carbon atoms, and still more preferably having 6 to 14 carbon atoms), a heterocyclic oxy group (preferably having 2 to 20 carbon atoms),

an alkoxycarbonyl group (preferably having 2 to 20 carbon atoms), a cycloalkoxycarbonyl group (preferably having 4 to 20 carbon atoms), an aryloxycarbonyl group (preferably having 6 to 20 carbon atoms), an amino group (preferably having 0 to 20 carbon atoms; the amino group includes an unsubstituted amino group (—NH₂), a (mono- or di-) alkylamino group, a (mono- or di-) alkenylamino group, a (mono- or di-) alkynylamino group, a (mono- or di-) cycloalkylamino group, a (mono- or di-) cycloalkenylamino group, a (mono- or di-) arylamino group, or a (mono- or di-) heterocyclic amino group, where each of the above groups substituting an unsubstituted amino group has the same definition as the corresponding group in the substituent group T), a sulfamoyl group (preferably having 0 to 20 carbon atoms; the sulfamoyl group is preferably an alkyl, cycloalkyl, or aryl sulfamoyl group), an acyl group (preferably having 1 to 20 carbon atoms, and more preferably having 2 to 15 carbon atoms), an acyloxy group (preferably having 1 to 20 carbon atoms), a carbamoyl group (preferably having 1 to 20 carbon atoms; the carbamoyl group is preferably an alkyl, cycloalkyl, or aryl carbamoyl group),

an acylamino group (preferably having 1 to 20 carbon atoms), an alkylthio group (preferably having 1 to 20 carbon atoms and more preferably 1 to 12 carbon atoms), a cycloalkylthio group (preferably having 3 to 20 carbon atoms), an arylthio group (preferably having 6 to 40 carbon atoms, more preferably 6 to 26 carbon atoms, and still more preferably 6 to 14 carbon atoms), a heterocyclic thio group (preferably having 2 to 20 carbon atoms), an alkyl, cycloalkyl, or aryl sulfonyl group (preferably having 1 to 20 carbon atoms),

a silyl group (preferably having 1 to 30 carbon atoms and more preferably 1 to 20 carbon atoms, which is preferably a silyl group at which an alkyl, aryl, alkoxy, or aryloxy has been substituted), a silyloxy group (preferably having 1 to 20 carbon atoms, which is preferably a silyloxy group at which an alkyl, aryl, alkoxy, or aryloxy has been substituted), a hydroxy group, a cyano group, a nitro group, a halogen atom (for example, a fluorine atom, a chlorine atom, a bromine atom or an iodine atom), an oxygen atom (specifically replacing >CH₂ which constitutes a ring with >C═O), a carboxy group (—CO₂H), a phosphono group [—PO(OH)₂], a phosphonooxy group [—O—PO(OH)₂], a sulfo group (—SO₃H), a boric acid group [—B(OH)₂], an onio group (an ammonio group including a cyclic ammonio group, which contains a sulfonio group(—SH₂⁺) or a phosphonio group (—PH3⁺), and preferably has 0 to 30 carbon atoms and more preferably 1 to 20 carbon atoms), a sulfanyl group (—SH), an amino acid residue, or a polyamino acid residue.

Further, examples thereof include a carboxy group, a phosphono group, a sulfo group, an onio group, an amino acid residue, or the above-described alkyl group, alkenyl group, alkynyl group, cycloalkyl group, cycloalkenyl group, aryl group, heterocycle group, alkoxy group, alkenyloxy group, alkynyloxy group, cycloalkyloxy group, aryloxy group, heterocyclic oxy group, alkoxycarbonyl group, cycloalkoxycarbonyl group, aryloxycarbonyl group, amino group, sulfamoyl group, acyl group, acyloxy group, carbamoyl group, acylamino group, alkylthio group, cycloalkylthio group, arylthio group, heterocyclic thio group, and an alkyl, cycloalkyl, or aryl sulfonyl group, where this above-described group has a polyamino acid residue as a substituent.

The substituent selected from the substituent group T is more preferably an alkyl group, an alkenyl group, a cycloalkyl group, an aryl group, a heterocycle group, an alkoxy group, a cycloalkoxy group, an aryloxy group, an alkoxycarbonyl group, a cycloalkoxycarbonyl group, an amino group, an acylamino group, a cyano group or a halogen atom, and particularly preferably an alkyl group, an alkenyl group, an aryl group, a heterocycle group, an alkoxy group, an alkoxycarbonyl group, an amino group, an acylamino group, or a cyano group.

The substituent selected from the substituent group T also includes a group obtained by combining a plurality of the above groups, unless otherwise specified. For example, in a case where a compound, a substituent, or the like contains an alkyl group, an alkenyl group, or the like, the alkyl group, the alkenyl group, or the like may be substituted or unsubstituted. In addition, in a case where a compound, a substituent, or the like contains an aryl group, a heterocycle group, or the like, the aryl group, the heterocycle group, or the like may be a monocyclic ring or a fused ring, and may be substituted or unsubstituted.

### EXAMPLES

Hereinafter, the present invention will be described in more detail based on Examples, but the present invention is not limited thereto. In the present invention, room temperature means 25° C.

The compounds used as Examples and Comparative Examples are shown below. In the following structural formulae, Me represents a methyl group. In carboxy groups in each of the compounds (10) to (12), it suffices that any one of the carboxy groups has a negative charge.

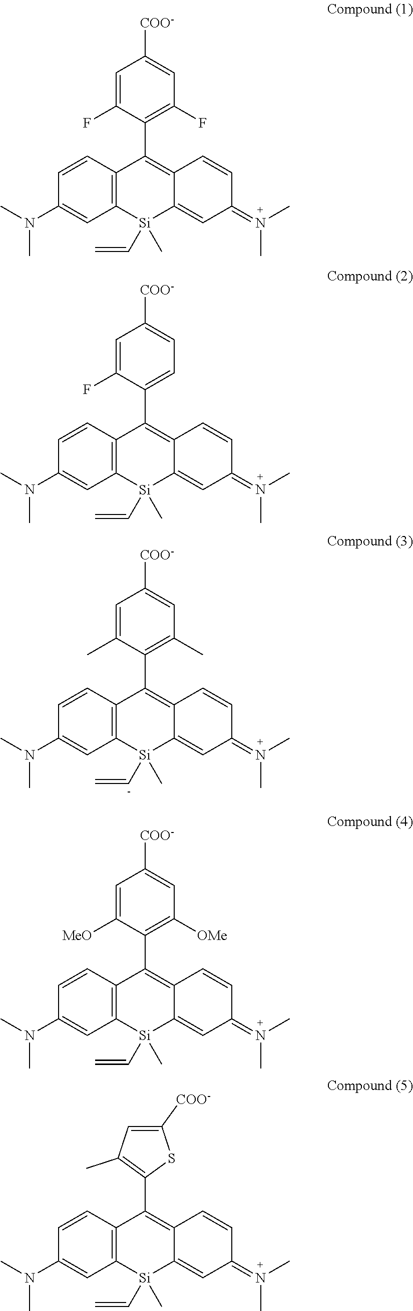

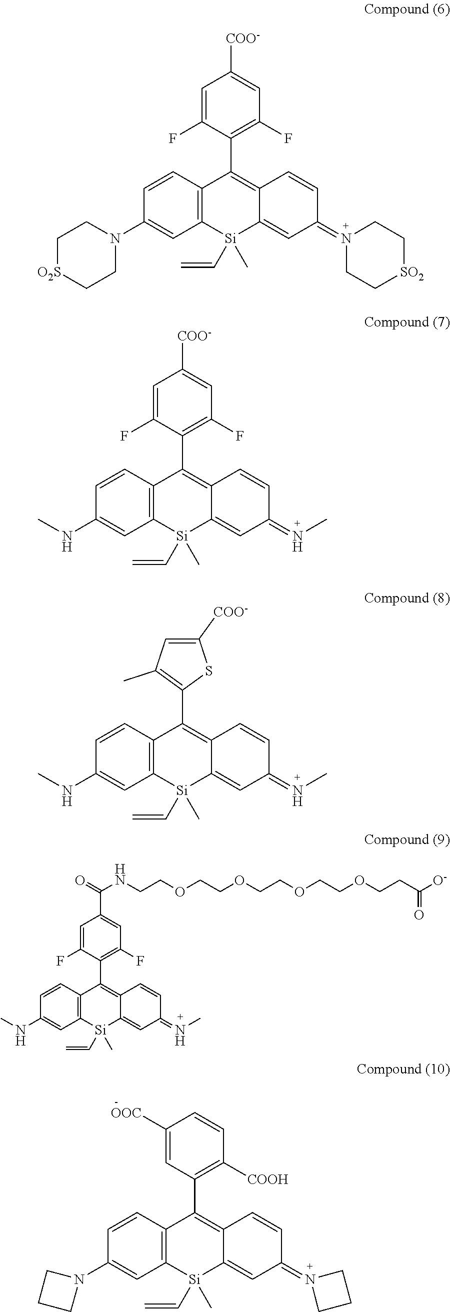

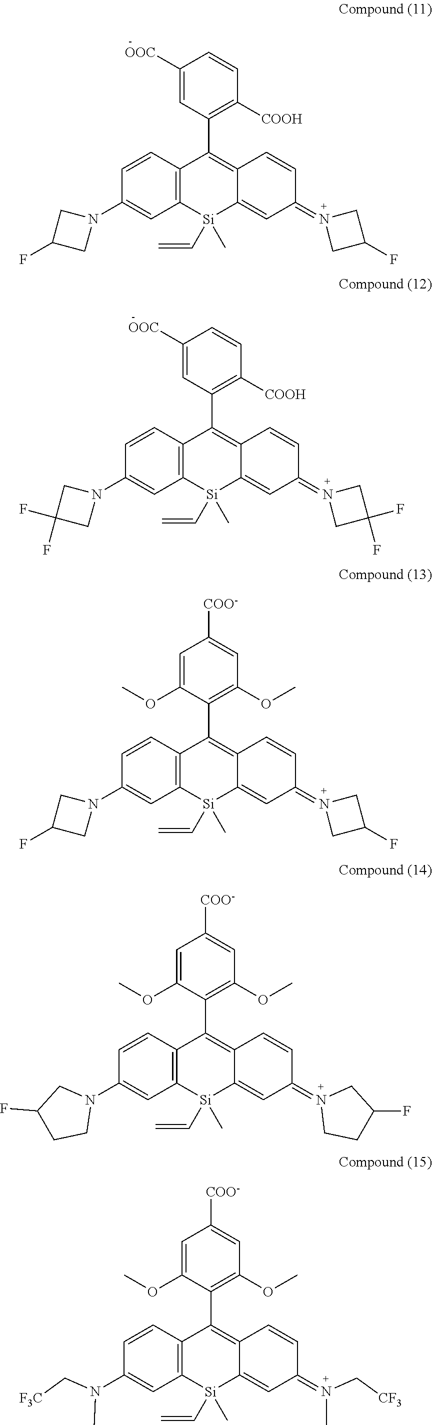

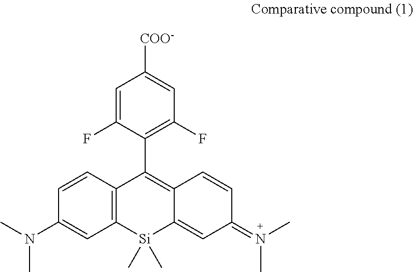

The methods of synthesizing a compound and a labeled antibody will be described in detail below; however, the starting materials, the dye intermediates, and the synthetic routes are not limited thereto.

Unless otherwise specified, as the carrier in the silica gel column chromatography, SNAP KP-Sil Cartridge (manufactured by Biotage, LLC) and High-Flash column W001, W002, W003, W004, or W005 [manufactured by Yamazen Corporation] were used. As NH silica, SNAP KP—NH Cartridge (manufactured by Biotage, LLC) was used. The mixing ratio in the eluent is based on a volume ratio. For example, “ethyl acetate:normal hexane=0:100→100:0” means that the eluent of “ethyl acetate:normal hexane=0:100” is changed to an eluent of “ethyl acetate:normal hexane=100:0”.

The MS spectrum was measured by ACQUITY SQD LC/MS System [manufactured by Waters Corporation, ionization method: electrospray Ionization (ESI)] or LCMS-2010EV [manufactured by Shimadzu Corporation, ionization method: an ionization method simultaneously carrying out ESI and atmospheric pressure chemical ionization (APCI)].

Unless otherwise specified, the synthesized compound and the labeled antibody used in each Example were those stored under the light-shielded conditions in a case where they were not used immediately after preparation. In addition, the commercially available compound and labeled antibody were stored under the light-shielded conditions after purchase until use.

The abbreviations used are summarized below.
DDQ: 2,3-dichloro-5,6-dicyano-1,4-benzoquinone
HSTU: O-(N-succinimidyl)-N,N,N′,N′-tetramethyluronium hexafluorophosphate
NMO: N-methylmorpholine-N-oxide
TBAF: Tetrabutylammonium fluoride
TPAP: Tetrapropylammonium perruthenate
TFA: Trifluoroacetic acid
X-phos: 2-dicyclohexylphosphino-2′,4′,6′-triisopropyl-1,1′-biphenyl
CPME: Cyclopentyl methyl ether
diF-azetidine-HCl: 3,3-difluoroazetidine hydrochloride
F-azetidine-HCl: 3-fluoroazetidine hydrochloride
F-pyrrolidine-HCl: 3-fluoropyrrolidine hydrochloride
DCM: Methylene chloride
DMF: N,N-dimethylformamide
DMSO: Dimethyl sulfoxide
THF: Tetrahydrofuran
Ac: Acetyl group
nBu: Normal butyl group
Et: Ethyl group
Me: Methyl group
PEG4: —(CH₂CH₂O)₄—
Ph: Phenyl group
iPr: Isopropyl group
TBS: Tert-butyldimethylsilyl group
Tf: Trifluoromethylsulfonyl group
dba: Dibenzylidene acetone

### Synthesis Example 1: Synthesis of Compound (1) and Labeled Antibody (1)

A compound (1) and a labeled antibody (1) were synthesized based on the following scheme.

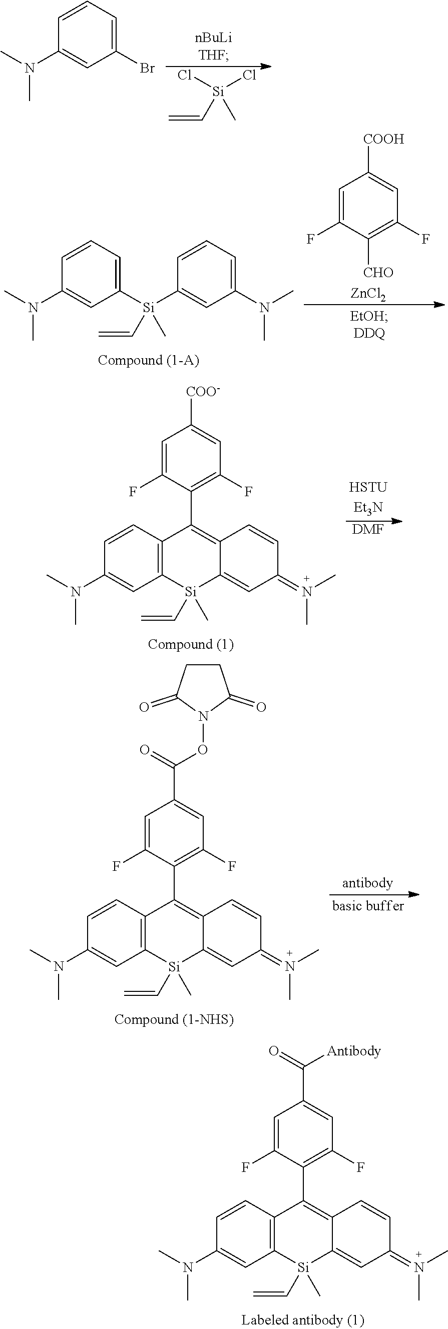

<Synthesis of Compound (1-A)>

3-bromo-N,N-dimethylaniline (1.00 g, manufactured by Fujifilm Wako Pure Chemical Corporation) and tetrahydrofuran (30 mL, ultra-dehydrated, manufactured by Fujifilm Wako Pure Chemical Corporation) were added in a 100 mL three-neck flask. After nitrogen substitution, cooling was carried out at 78° C., n-butyllithium (3.17 mL, 1.6 M, an n-hexane solution, manufactured by Kanto Chemical Co., Inc.) was added dropwise thereto, and stirring was carried out for 30 minutes. Subsequently, dichloromethylvinylsilane (0.309 mL, manufactured by Tokyo Chemical Industry Co., Ltd.) was added thereto, the temperature was raised to room temperature, and stirring was carried out for 5 minutes. The completion of the reaction was confirmed by LC/MS, a saturated aqueous solution of ammonium chloride was added under ice-cooling thereto, extraction was carried out with ethyl acetate, drying was carried out with a Glauber's salt, and the solvent was distilled off under reduced pressure. The obtained residue was dissolved in methylene chloride and purified by silica gel column chromatography (ethyl acetate:normal hexane=0:100→10:90), a product of interest was recovered, and the solvent was distilled off under reduced pressure to obtain a colorless to yellow oily compound (1-A) (0.62 g, yield: 83%).

[M+H⁺]+: 311

<Synthesis of Compound (1)>

The compound (1-A) (58 mg), 3,5-difluoro-4-formylbenzoic acid (35 mg, manufactured by COMBI-BLOCKS Inc.), zinc (II) chloride (76 mg, manufactured by FUJIFILM Wako Pure Chemical Corporation), and ethanol (0.64 mL, ultra-dehydrated, manufactured by FUJIFILM Wako Pure Chemical Corporation) were added in a 5 mL test tube type reaction container, irradiated with a microwave, and heated at 140° C. for 30 minutes. The completion of the reaction was confirmed by LC/MS, and the solvent was distilled off under reduced pressure. Methylene chloride (1.85 mL, manufactured by Fujifilm Wako Pure Chemical Corporation) was added to the obtained residue, 2,3-dichloro-5,6-dicyano-1,4-benzoquinone (42 mg) was subsequently added thereto under ice-cooling, the temperature was raised to room temperature, and then stirring was carried out for 5 minutes. The completion of the reaction was confirmed by LC/MS, the solvent was distilled off under reduced pressure, purification was carried out by reverse phase silica gel column chromatography (C18, manufactured by Biotage, LLC, acetonitrile:distilled water=0:100→60:40) with a solution to which acetonitrile was added, a product of interest was recovered, and the solvent was distilled off by lyophilization. The obtained residue was dissolved in DMSO, purification was carried out by preparative HPLC (manufactured by Waters Corporation, acetonitrile:0.1% ammonium formate-containing aqueous solution=10:90→90:10), a product of interest was recovered, and the solvent was distilled off by lyophilization to obtain a dark blue powder compound (1) (3 mg, yield: 3%).

[M+H⁺]+: 477

<Synthesis of Compound (1-NHS)>

The compound (1) (1 mg), 0-(N-succinimidyl)-N,N,N′,N′-tetramethyluronium hexafluorophosphate (2 mg, manufactured by Tokyo Chemical Industry Co., Ltd.), triethylamine (2 μL, manufactured by Fujifilm Wako Pure Chemical Corporation), and DMF (100 μL, manufactured by Fujifilm Wako Pure Chemical Corporation) were added in a 2 mL test tube type reaction container, and stirring was carried out at room temperature for 2 hours. The completion of the reaction was confirmed by LC/MS, the reaction solution was directly purified by reverse phase silica gel column chromatography (C18, manufactured by Biotage, LLC, acetonitrile:distilled water=0:100→60:40), a product of interest was recovered, and the solvent was distilled off by lyophilization to obtain a dark blue powder compound (1-NHS) (0.5 mg, yield: 41%).

[M+]⁺: 574

<Synthesis of Labeled Antibody (1)>

40 μL of a carbonate pH standard solution (pH=10.01) (FUJIFILM Wako Pure Chemical Corporation) and 3.2 μL of a DMSO solution having a concentration of the compound (1-NHS) of 20 mM were added to 400 μL of an anti-rabbit IgG antibody [host: goat, 2.4 mg/mL, catalog number: 111-005-003, manufactured by Jackson ImmunoResearch Inc.], and the resultant mixture was stirred and allowed to stand at room temperature for 1 hour. Subsequently, the reaction solution was directly applied onto a Sephadex G-25 column [catalog number: 17085101, manufactured by GE Healthcare] and purified by using PBS [pH=7.4, manufactured by FUJIFILM Wako Pure Chemical Corporation], thereby obtaining the labeled antibody (1).

### Synthesis Example 2: Synthesis of Compound (2) and Labeled Antibody (2)

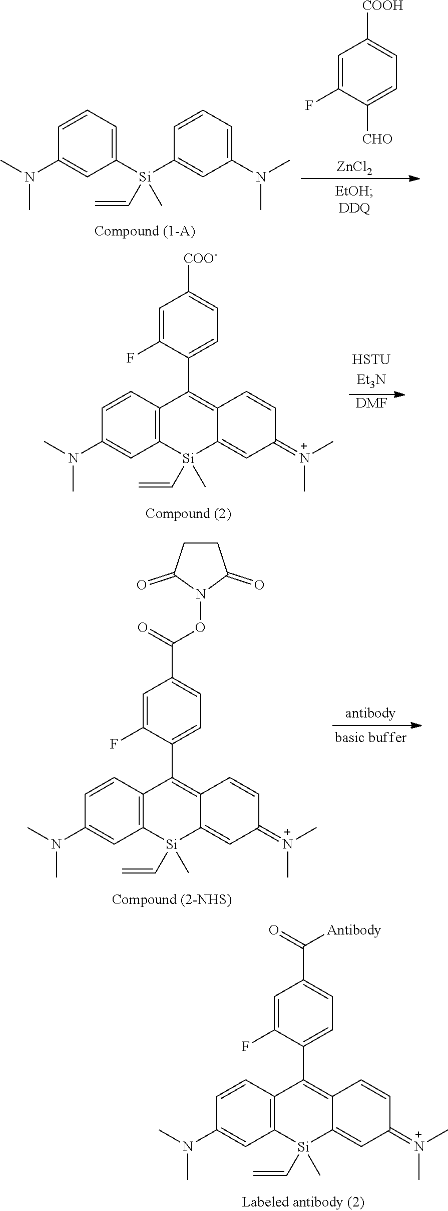

<Synthesis of Compound (2)>

2 mg of a dark blue powder compound (2) was obtained in the same manner, except that in the synthesis method for the compound (1), 3-fluoro-4-formylbenzoic acid (manufactured by ChemExpress Co., Ltd.) was used instead of 3,5-difluoro-4-formylbenzoic acid.

[M+H⁺]⁺: 459

<Synthesis of Compound (2-NHS)>

except that in the synthesis method for the compound (1-NHS), the compound (2) was used instead of the compound (1).0.5 mg of a dark blue powder compound (2-NHS) was obtained in the same manner,

[M+]⁺: 556

<Synthesis of Labeled Antibody (2)>

A labeled antibody (2) was obtained in the same manner except that in the synthesis method for the labeled antibody (1), the compound (2-NHS) was used instead of the compound (1-NHS).

### Synthesis Example 3: Synthesis of Compound (3) and Labeled Antibody (3)

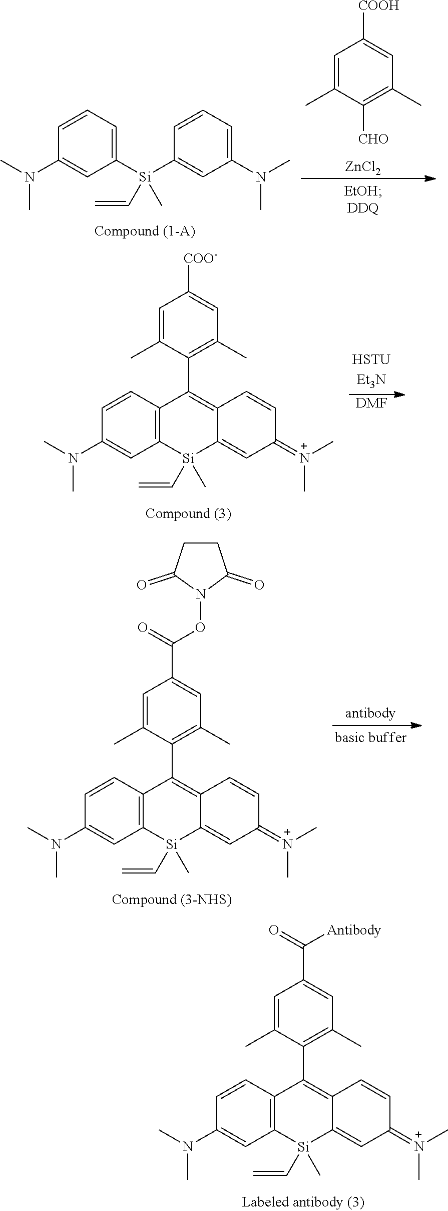

<Synthesis of Compound (3)>

1 mg of a dark blue powder compound (5) was obtained in the same manner, except that in the synthesis method of the compound (1), 4-formyl-3.5-dimethylbenzoic acid (manufactured by AOBChem) was used instead of 3,5-difluoro-4-formylbenzoic acid.

[M+H⁺]⁺: 469

<Synthesis of Compound (3-NHS)>

0.2 mg of a dark blue powder compound (3-NHS) was obtained in the same manner, except that in the synthesis method for the compound (1-NHS), the compound (3) was used instead of the compound (1).

[M+]⁺: 566

<Synthesis of Labeled Antibody (3)>

A labeled antibody (3) was obtained in the same manner except that in the synthesis method for the labeled antibody (1), the compound (3-NHS) was used instead of the compound (1-NHS).

### Synthesis Example 4: Synthesis of Compound (4) and Labeled Antibody (4)

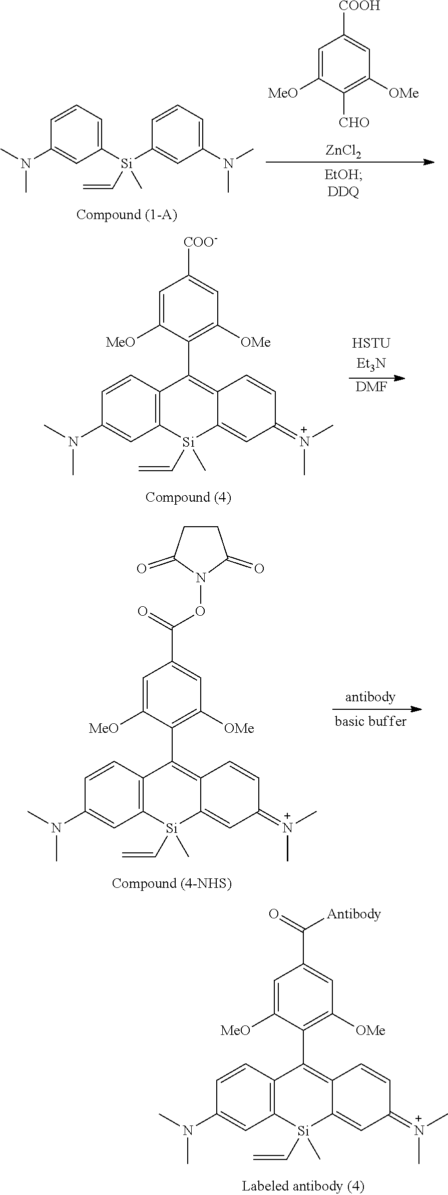

<Synthesis of Compound (4)>

2 mg of a dark blue powder compound (4) was obtained in the same manner, except that in the synthesis method for the compound (1), 4-formyl-3,5-dimethoxybenzoic acid (manufactured by COMBI-BLOCKS Inc.) was used instead of 3,5-difluoro-4-formyl benzoic acid.

[M+H⁺]⁺: 501

<Synthesis of Compound (4-NHS)>

0.4 mg of a dark blue powder compound (4-NHS) was obtained in the same manner, except that in the synthesis method for the compound (1-NHS), the compound (4) was used instead of the compound (1).

[M+]⁺: 598

<Synthesis of Labeled Antibody (4)>

A labeled antibody (4) was obtained in the same manner except that in the synthesis method for the labeled antibody (1), the compound (4-NHS) was used instead of the compound (1-NHS).

### Synthesis Example 5: Synthesis of Compound (5) and Labeled Antibody (5)

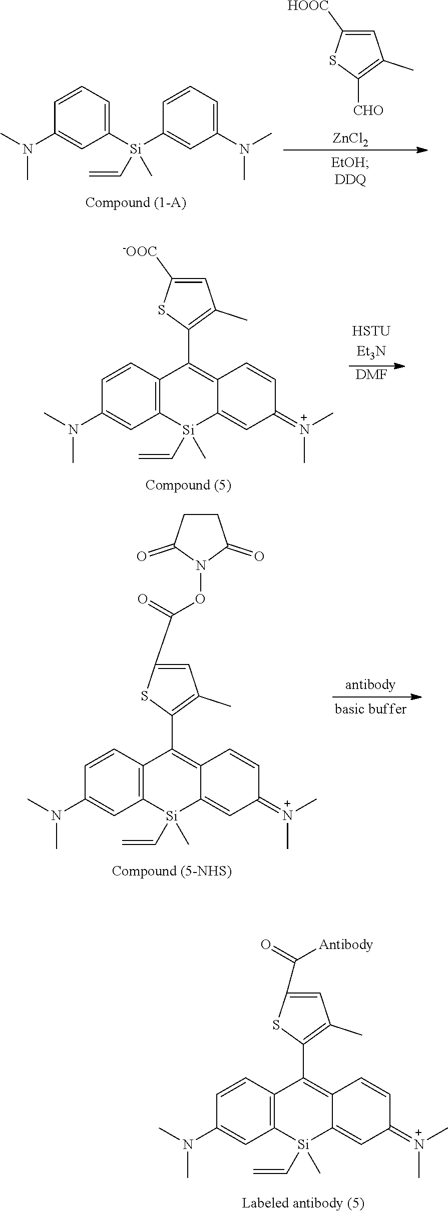

<Synthesis of Compound (5)>

1 mg of a dark blue powder compound (5) was obtained in the same manner, except that in the synthesis method for the compound (1), 5-formyl-4-methylthiophene-2-carboxylic acid (manufactured by Enamine Ltd.) was used instead of 3,5-difluoro-4-formylbenzoic acid.

[M+H⁺]⁺: 461

<Synthesis of Compound (5-NHS)>

0.3 mg of a dark blue powder compound (5-NHS) was obtained in the same manner, except that in the synthesis method for the compound (1-NHS), the compound (5) was used instead of the compound (1).

[M+]⁺: 558

<Synthesis of Labeled Antibody (5)>

A labeled antibody (5) was obtained in the same manner except that in the synthesis method for the labeled antibody (1), the compound (5-NHS) was used instead of the compound (1-NHS).

### Synthesis Example 6: Synthesis of Compound (6) and Labeled Antibody (6)

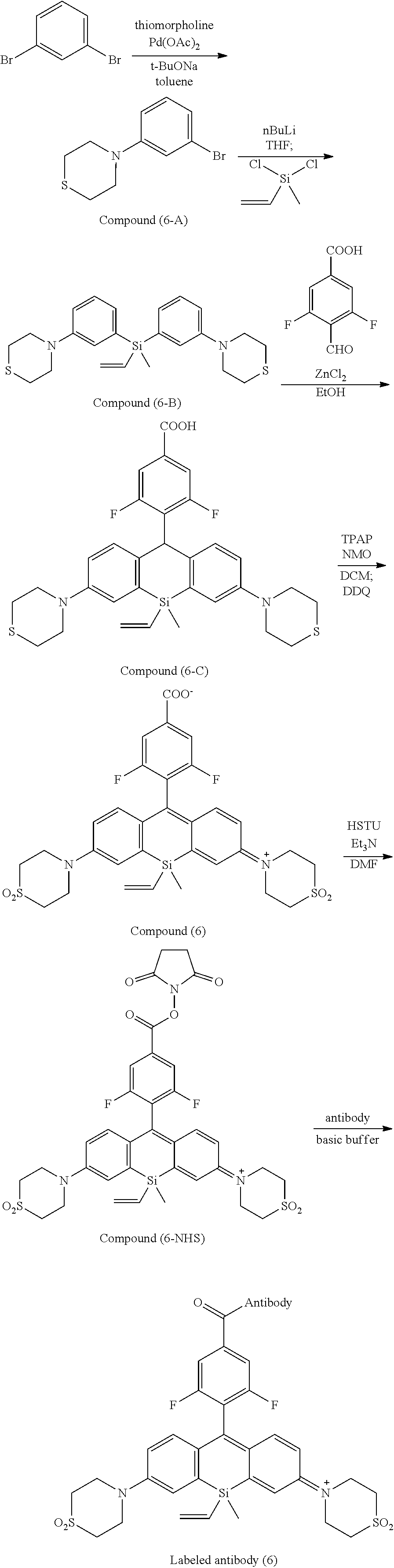

<Synthesis of Compound (6-A)>

1,3-dibromobenzene (1.89 g, manufactured by Fujifilm Wako Pure Chemical Corporation), thiomorpholine (0.83 g, manufactured by Fujifilm Wako Pure Chemical Corporation), palladium acetate (0.09 g, FUJIFILM Wako Pure Chemical Corporation), BINAP (0.37 g, racemic body, FUJIFILM Wako Pure Chemical Corporation), sodium t-butoxide (0.92 g, manufactured by FUJIFILM Wako Pure Chemical Corporation), and toluene (10 mL, manufactured by FUJIFILM Wako Pure Chemical Corporation) were added in a 20 mL test tube type reaction container, irradiated with a microwave, and stirred at 140° C. for 45 minutes. The completion of the reaction was confirmed by LC/MS, the reaction solution was directly purified by silica gel column chromatography (ethyl acetate:normal hexane=0:100→20:80), a product of interest was recovered, and the solvent was distilled off under reduced pressure to obtain a colorless oily compound (6-A) (1.22 g, yield: 59%).

[M+H⁺]⁺: 258, 260

<Synthesis of Compound (6-B)>

A yellow oily compound (6-B) (471 mg, yield: 26%) was obtained in the same manner, except that in the synthesis method for the compound (1-A), the compound (6-A) (2.30 g) was used instead of 3-bromo-N,N-dimethylaniline.

[M+H⁺]⁺: 427

<Synthesis of Compound (6-C)>

The compound (6-B) (50 mg), 3,5-difluoro-4-formylbenzoic acid (65 mg, manufactured by COMBI-BLOCKS Inc.), zinc (II) chloride (48 mg, manufactured by FUJIFILM Wako Pure Chemical Corporation), and ethanol (0.47 mL, ultra-dehydrated, manufactured by FUJIFILM Wako Pure Chemical Corporation) were added in a 5 mL test tube type reaction container, irradiated with a microwave, and heated at 140° C. for 30 minutes. The completion of the reaction was confirmed by LC/MS, and the solvent was distilled off under reduced pressure. Methylene chloride (1.85 mL, manufactured by Fujifilm Wako Pure Chemical Corporation) was added to the obtained residue, purification was carried out by silica gel column chromatography (ethyl acetate:normal hexane=0:100→50:50), a product of interest was recovered, and the solvent was distilled off under reduced pressure to obtain a yellow to light blue oily compound (6-C) (50 mg, yield: 72%).

[M+H⁺]⁺: 595

<Synthesis of Compound (6)>

The compound (6-C) (24 mg), tetrapropylammonium perruthenate (7 mg, manufactured by FUJIFILM Wako Pure Chemical Corporation), N-methylmorpholine-N-oxide (118 mg, FUJIFILM Wako Pure Chemical Corporation), and methylene chloride (1.01 mL, manufactured by FUJIFILM Wako Pure Chemical Corporation) were added in a 5 mL test tube type reaction container and stirred at 80° C. for 20 minutes. The completion of the reaction was confirmed by LC/MS, 2,3-dichloro-5,6-dicyano-1,4-benzoquinone (24 mg) was subsequently added thereto under ice-cooling, the temperature was raised to room temperature, and then stirring was carried out for 5 minutes. The completion of the reaction was confirmed by LC/MS, the solvent was distilled off under reduced pressure, purification was carried out by reverse phase silica gel column chromatography (C18, manufactured by Biotage, LLC, acetonitrile:distilled water=0:100→60:40) with a solution to which acetonitrile was added, and a product of interest was recovered, a product of interest was recovered, and the solvent was distilled off by lyophilization to obtain a dark blue powder compound (6) (0.4 mg, yield: 2%).

[M+H⁺]⁺: 657

<Synthesis of Compound (6-NHS)>

0.1 mg of a dark blue powder compound (6-NHS) was obtained in the same manner, except that in the synthesis method for the compound (1-NHS), the compound (6) was used instead of the compound (1).

[M⁺]⁺: 754

<Synthesis of Labeled Antibody (6)>

A labeled antibody (6) was obtained in the same manner except that in the synthesis method for the labeled antibody (1), the compound (6-NHS) was used instead of the compound (1-NHS).

### Synthesis Example 7: Synthesis of Compound (7) and Labeled Antibody (7)

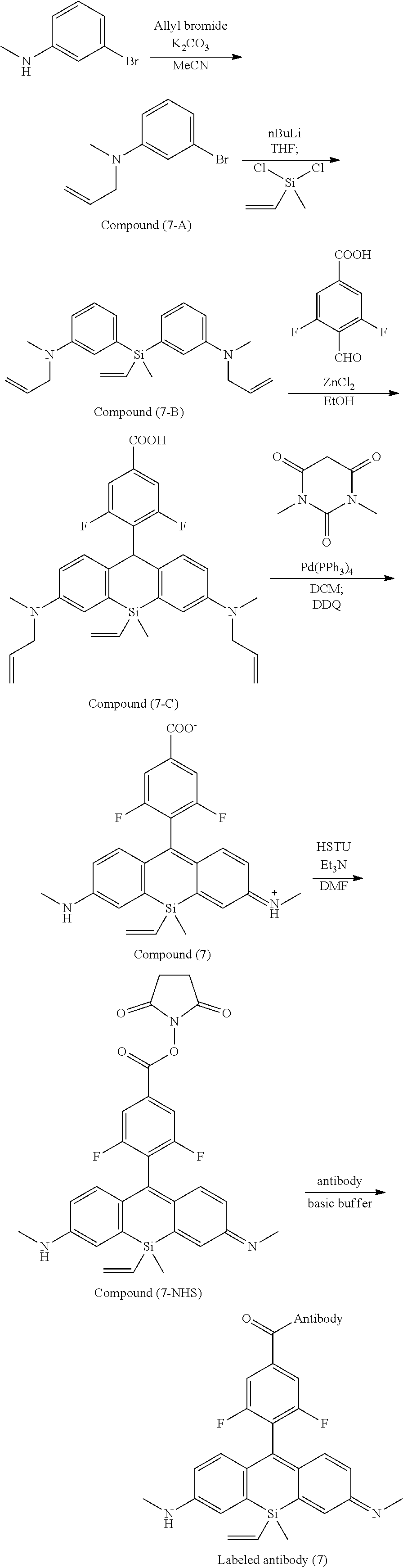

<Synthesis of Compound (7-A)>

3-bromo-N-methylaniline (5.00 g, manufactured by Fujifilm Wako Pure Chemical Corporation), allyl bromide (2.80 mL, manufactured by Fujifilm Wako Pure Chemical Corporation), potassium carbonate (7.50 g, FUJIFILM Wako Pure Chemical Corporation), and acetonitrile (25 mL, manufactured by FUJIFILM Wako Pure Chemical Corporation) were added in a 100 mL eggplant flask and stirred at 90° C. for 16 hours. The completion of the reaction was confirmed by LC/MS, the insoluble matter was removed by filtration, and then the solvent was distilled off under reduced pressure. The obtained residue was prepared as a solution with n-hexane and purified by silica gel column chromatography (ethyl acetate: normal hexane=0:100→10:90), a product of interest was recovered, and the solvent was distilled off under reduced pressure to obtain a yellow oily compound (7-A) (5.57 g, yield: 92%).

[M+H⁺]⁺: 226, 228

<Synthesis of Compound (7-B)>

A yellow oily compound (7-B) (1.51 g, yield: 98%) was obtained in the same manner, except that in the synthesis method for the compound (1-A), the compound (7-A) (2.01 g) was used instead of 3-bromo-N,N-dimethylaniline.

[M+H⁺]⁺: 363

<Synthesis of Compound (7-C)>

A yellow to light blue oily compound (7-C) (109 mg, yield: 25%) was obtained in the same manner, except that in the synthesis method for the compound (6-C), the compound (7-B) (300 mg) was used instead of the compound (6-B).

<Synthesis of Compound (7)>

The compound (7-C) (109 mg), barbituric acid (481 mg, manufactured by Fujifilm Wako Pure Chemical Corporation), tetrakistriphenylphosphine palladium (48 mg, manufactured by Fujifilm Wako Pure Chemical Corporation), and methylene chloride (11 mL, FUJIFILM Wako Pure Chemical Corporation) were added in a 50 mL eggplant flask and stirred at room temperature for 30 minutes. The completion of the reaction was confirmed by LC/MS, 2,3-dichloro-5,6-dicyano-1,4-benzoquinone (50 mg) was subsequently added thereto under ice-cooling, the temperature was raised to room temperature, and then stirring was carried out for 5 minutes. The completion of the reaction was confirmed by LC/MS, the solvent was distilled off under reduced pressure, purification was carried out by reverse phase silica gel column chromatography (C18, manufactured by Biotage, LLC, acetonitrile:distilled water=0:100→60:40) with a solution to which acetonitrile was added, and a product of interest was recovered, and the solvent was distilled off by lyophilization to obtain a dark blue powder compound (7) (5 mg, yield: 6%).

[M+H⁺]⁺: 449

<Synthesis of Compound (7-NHS)>

2 mg of a dark blue powder compound (7-NHS) was obtained in the same manner, except that in the synthesis method for the compound (1-NHS), the compound (7) was used instead of the compound (1).

[M+H⁺]⁺: 546

<Synthesis of Labeled Antibody (7)>

A labeled antibody (7) was obtained in the same manner except that in the synthesis method for the labeled antibody (1), the compound (7-NHS) was used instead of the compound (1-NHS).

### Synthesis Example 8: Synthesis of Compound (8) and Labeled Antibody (8)

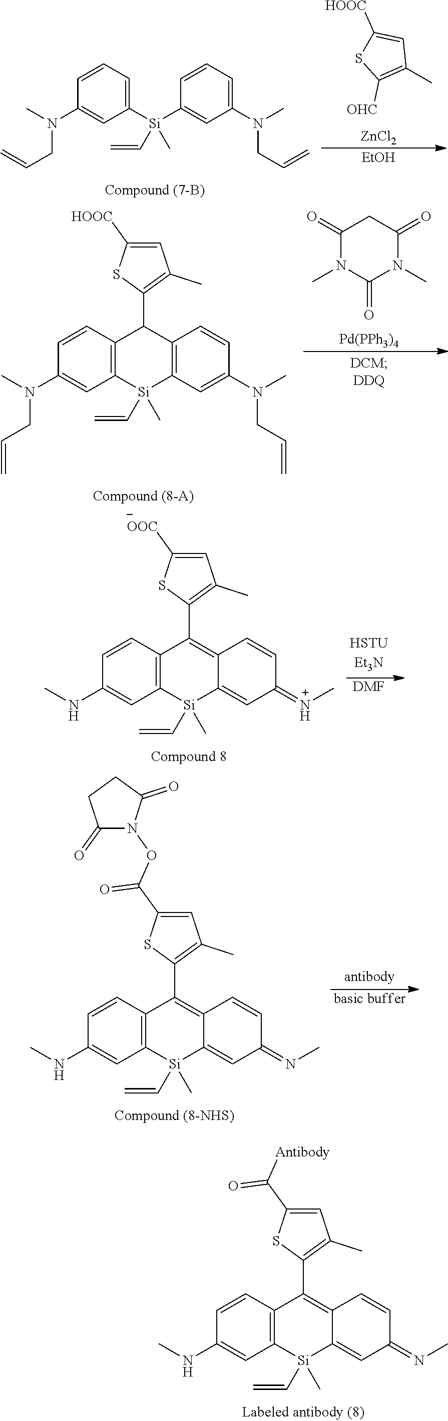

<Synthesis of Compound (8-A)>

A yellow oily compound (8-A) (56 mg, yield: 13%) was obtained in the same manner, except that in the synthesis method for the compound (7-C), 5-formyl-4-methylthiophene-2-carboxylic acid (manufactured by Enamine Ltd.) was used instead of 3,5-difluoro-5-formylbenzoic acid.

[M+H⁺]⁺: 515

<Synthesis of Compound (8)>

A dark blue powder compound (8) (1 mg, yield: 1%) was obtained in the same manner, except that in the synthesis method for the compound (7), the compound (8-A) was used instead of the compound (7-C).

[M+H⁺]⁺: 433

<Synthesis of Compound (8-NHS)>

0.1 mg of a dark blue powder compound (8-NHS) was obtained in the same manner, except that in the synthesis method for the compound (7-NHS), the compound (8) was used instead of the compound (7).

[M+H⁺]⁺: 530

<Synthesis of Labeled Antibody (8)>

A labeled antibody (8) was obtained in the same manner except that in the synthesis method for the labeled antibody (7), the compound (8-NHS) was used instead of the compound (7-NHS).

### Synthesis Example 9: Synthesis of Compound (9) and Labeled Antibody (9)

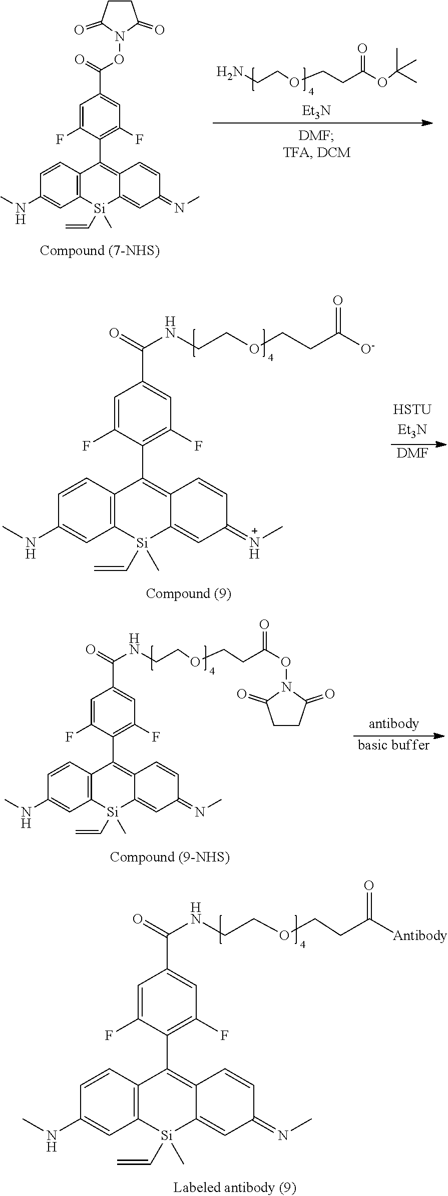

<Synthesis of Compound (9)>

The compound (7-NHS) (1 mg), amino-PEG4-t-butyl ester (2 mg, manufactured by Tokyo Chemical Industry Co., Ltd.), triethylamine (1 μL, manufactured by Fujifilm Wako Pure Chemical Corporation), DMF (200 μL, FUJIFILM Wako Pure Chemical Corporation) were added in a 2 mL test tube type reaction container and stirred at room temperature for 16 hours. The completion of the reaction was confirmed by LC/MS, the solvent was distilled off under reduced pressure, and methylene chloride (400 μL, manufactured by FUJIFILM Wako Pure Chemical Corporation) and trifluoroacetic acid (100 μL, FUJIFILM Wako Pure Chemical Corporation) were added to the obtained residue, followed by stirring at room temperature for 2 hours. The completion of the reaction was confirmed by LC/MS, purification was carried out by reverse phase silica gel column chromatography (C18, manufactured by Biotage, LLC, acetonitrile:distilled water=0:100→60:40) with a solution to which acetonitrile was added, a product of interest was recovered, and the solvent was distilled off by lyophilization to obtain a dark blue powder compound (9) (1 mg, yield: 79%).

[M+H⁺]⁺: 696

<Synthesis of Compound (9-NHS)>

0.2 mg of a dark blue powder compound (9-NHS) was obtained in the same manner, except that in the synthesis method for the compound (7-NHS), the compound (9) was used instead of the compound (7).

[M+H⁺]⁺: 793

<Synthesis of Labeled Antibody (9)>

A labeled antibody (9) was obtained in the same manner except that in the synthesis method for the labeled antibody (7), the compound (9-NHS) was used instead of the compound (7-NHS).

### Synthesis Example 10: Synthesis of Compound (10) and Labeled Antibody (10)

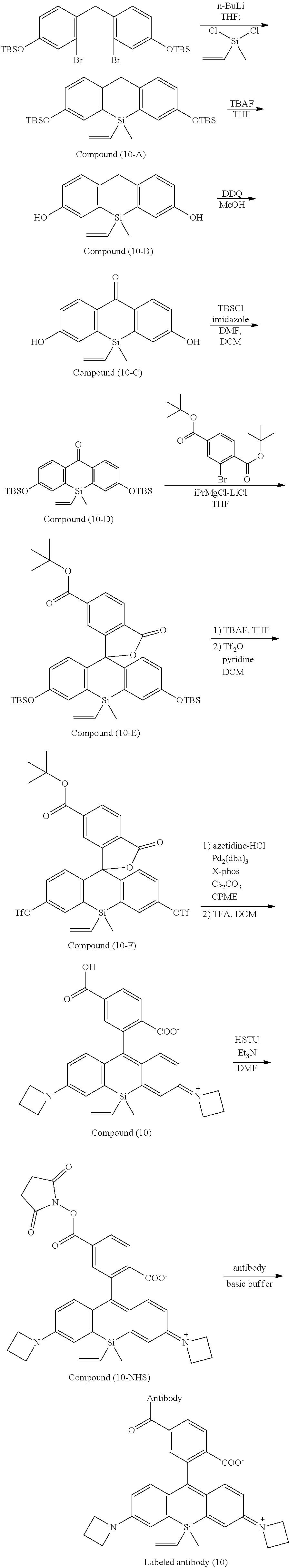

<Synthesis of Compound (10-A) to Compound (10-NHS)>

A dark blue powder compound (10) (2 mg) and a compound (10-NHS) (0.5 mg) were obtained in the same manner, except that in the synthesis method for the compound example 33 (2-(3,7-di(azetidine-1-yl)-5,5-dimethyldibenzo[b,e]cillin-10-ylium-10 (5H)-yl)-4-(((2,5-dioxopyrrolidine-1-yl)oxy)carbonyl)benzoate) in US2018/0284105A, dichloromethylvinylsilane (manufactured by Tokyo Chemical Industry Co., Ltd.) was used instead of dichlorodimethylsilane.

Compound (10)

[M+H⁺]⁺: 509

Compound (10-NHS)

[M+H⁺]⁺: 606

<Synthesis of Labeled Antibody (10)>

A labeled antibody (10) was obtained in the same manner except that in the synthesis method for the labeled antibody (1), the compound (10-NHS) was used instead of the compound (1-NHS).

### Synthesis Example 11: Synthesis of Compound (11) and Labeled Antibody (11)

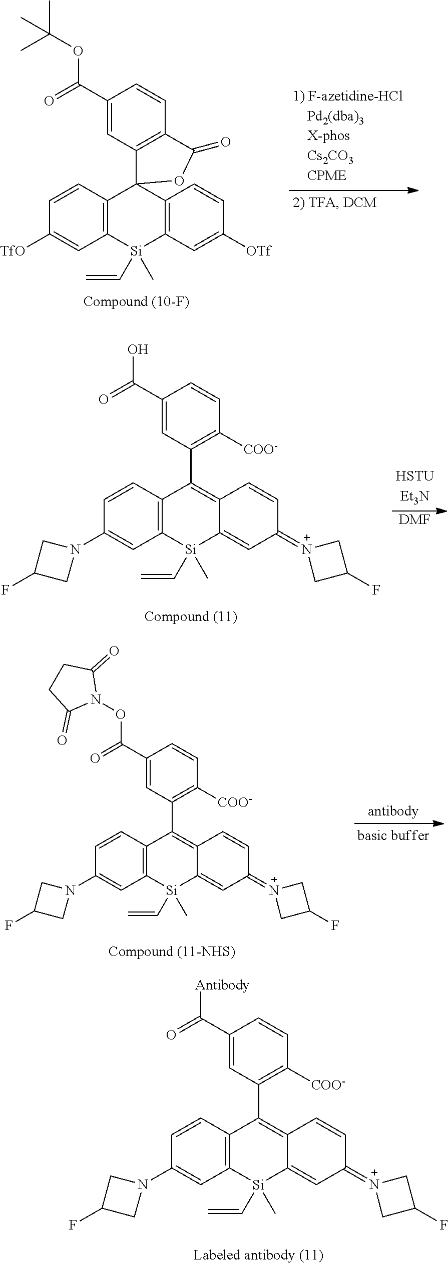

<Synthesis of Compound (11) and Compound (11-NHS)>

A compound (11) (1 mg) and a compound (11-NHS) (0.3 mg) were obtained in the same manner, except that in the synthesis method for the compound (10) and the compound (10-A), 3-fluoroazetidine hydrochloride (manufactured by Tokyo Chemical Industry Co., Ltd.) was used instead of azetidine hydrochloride.

Compound (11)

[M+H⁺]⁺: 545

Compound (11-NHS)

[M+H⁺]⁺: 642

<Synthesis of Labeled Antibody (11)>

A labeled antibody (11) was obtained in the same manner except that in the synthesis method for the labeled antibody (1), the compound (11-NHS) was used instead of the compound (1-NHS).

### [Synthesis Example 12: Synthesis of Compound (12) and Labeled Antibody (12)]

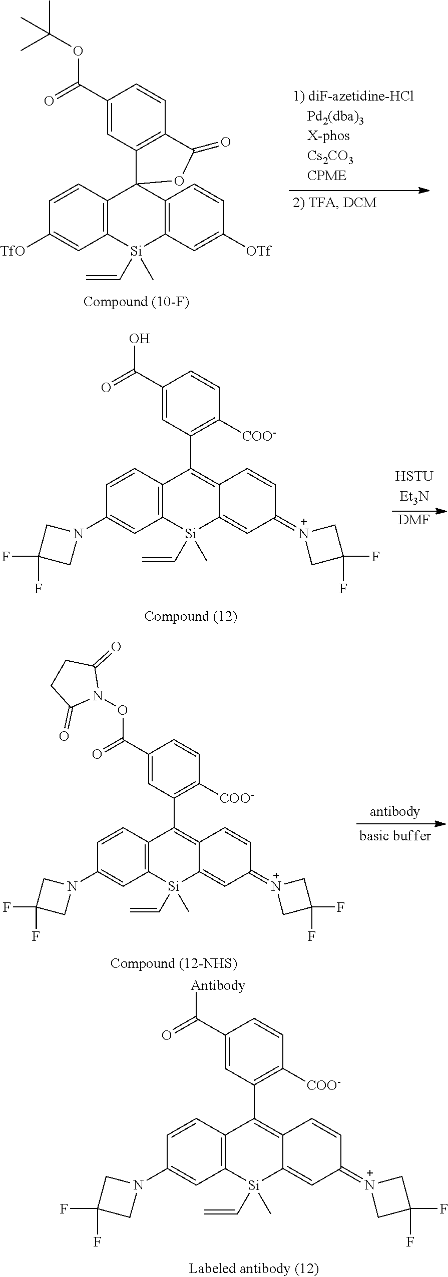

<Synthesis of Compound (12) and Compound (12-NHS)>

A compound (12) (0.6 mg) and a compound (12-NHS) (0.1 mg) were obtained in the same manner, except that in the synthesis method for the compound (10) and the compound (10-A), 3,3-difluoroazetidine hydrochloride (manufactured by FUJIFILM Wako Pure Chemical Corporation) was used instead of azetidine hydrochloride.

Compound (12)

[M+H⁺]⁺: 581

Compound (12-NHS)

[M+H⁺]⁺: 678

<Synthesis of Labeled Antibody (12)>

A labeled antibody (12) was obtained in the same manner except that in the synthesis method for the labeled antibody (1), the compound (12-NHS) was used instead of the compound (1-NHS).

### [Synthesis Example 13: Synthesis of Compound (13) and Labeled Antibody (13)]

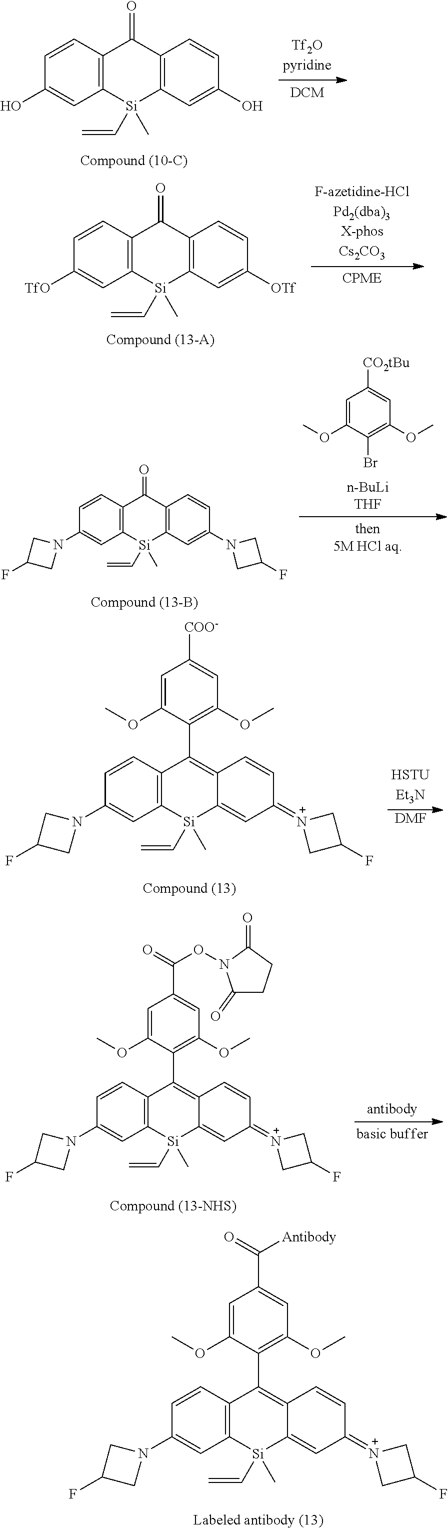

<Synthesis of Compound (13-A)>

A white powder compound (13-A) was obtained in the same manner, except that in the synthesis method for the compound (10-F), the compound (10-C) was used instead of the compound (10-E).

<Synthesis of Compound (13-B)>

A yellow powder compound (13-B) (414 mg, yield: 62%) was obtained in the same manner, except that in the synthesis method for the compound (10), the compound (13-A) was used instead of the compound (10-F) and 3-fluoroazetidine hydrochloride was used instead of azetidine hydrochloride.

[M+H⁺]⁺: 397

<Synthesis of Compound (13)>

200 mg of tert-butyl-4-bromo-3,5-dimethoxybenzoic acid (manufactured by Combi-Blocks Inc.) and 2 mL of tetrahydrofuran were added in a 10 mL eggplant flask, and after nitrogen substitution, cooling was carried out at 78° C., n-butyllithium (0.405 mL, 1.6 M, an n-hexane solution, manufactured by Kanto Chemical Co., Inc.) was added dropwise thereto, and stirring was carried out for 15 minutes. Subsequently, a tetrahydrofuran solution (3 mL) of 51 mg of the compound (13-B) was added thereto, and the temperature was raised to −10° C. while carrying out stirring. The completion of the reaction was confirmed by LC/MS, and an aqueous solution of 5 M hydrochloric acid was added thereto. After distilling off the solvent under reduced pressure, the obtained residue was purified by reverse phase silica gel column chromatography (C18, manufactured by Biotage, LLC, acetonitrile:distilled water containing 0.1% trifluoroacetic acid=5:95→50:50), a product of interest was recovered, and the solvent was distilled off by lyophilization to obtain a dark blue powder compound (13) (37 mg, yield: 44%).

[M+H⁺]⁺: 561

<Synthesis of Compound (13-NHS)>

A dark blue powder compound (13-NHS) was obtained in the same manner, except that in the synthesis method for the compound (1-NHS), the compound (13) was used instead of the compound (1).

<Synthesis of Labeled Antibody (13)>

A labeled antibody (13) was obtained in the same manner except that in the synthesis method for the labeled antibody (1), the compound (13-NHS) was used instead of the compound (1-NHS).

### [Synthesis Example 14: Synthesis of Compound (14) and Labeled Antibody (14)]

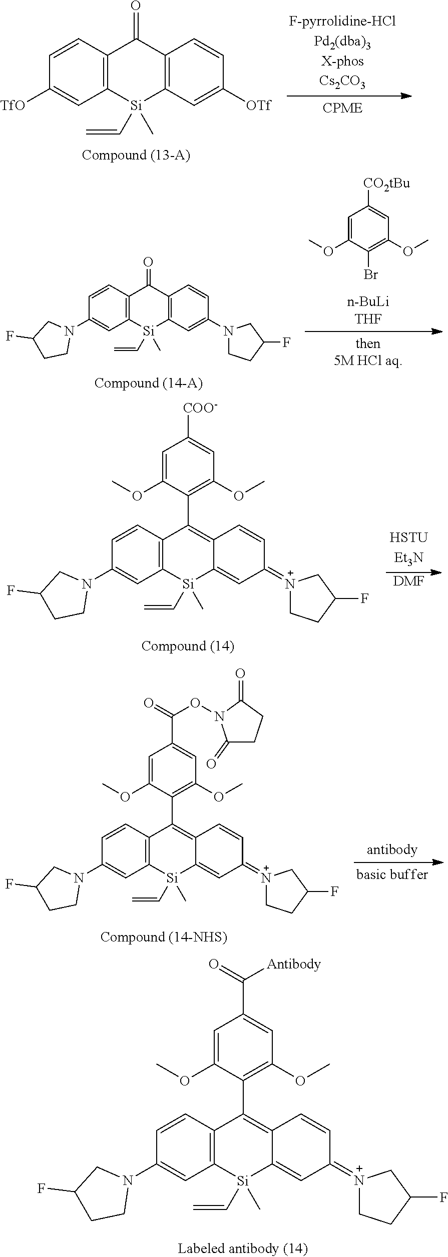

<Synthesis of Compound (14-A)>

except that in the synthesis method for the compound (13-B), 3-fluoroyrrolidine hydrochloride (manufactured by Enamine Ltd.) was used instead of 3-fluoroazetidine hydrochloride. A yellow powder compound (14-A) (213 mg, yield: 47%) was obtained in the same manner,

[M+H⁺]⁺: 425

<Synthesis of Compound (14)>

A dark blue powder compound (14) (3.5 mg, yield: 5.6%) was obtained in the same manner, except that in the synthesis method for the compound (13), the compound (14-A) was used instead of the compound (13-B).

[M+H⁺]⁺: 589

<Synthesis of Compound (14-NHS)>

A dark blue powder compound (14-NHS) was obtained in the same manner, except that in the synthesis method for the compound (1-NHS), the compound (14) was used instead of the compound (1).

<Synthesis of Labeled Antibody (14)>

A labeled antibody (14) was obtained in the same manner except that in the synthesis method for the labeled antibody (1), the compound (14-NHS) was used instead of the compound (1-NHS).

### [Synthesis Example 15: Synthesis of Compound (15) and Labeled Antibody (15)]

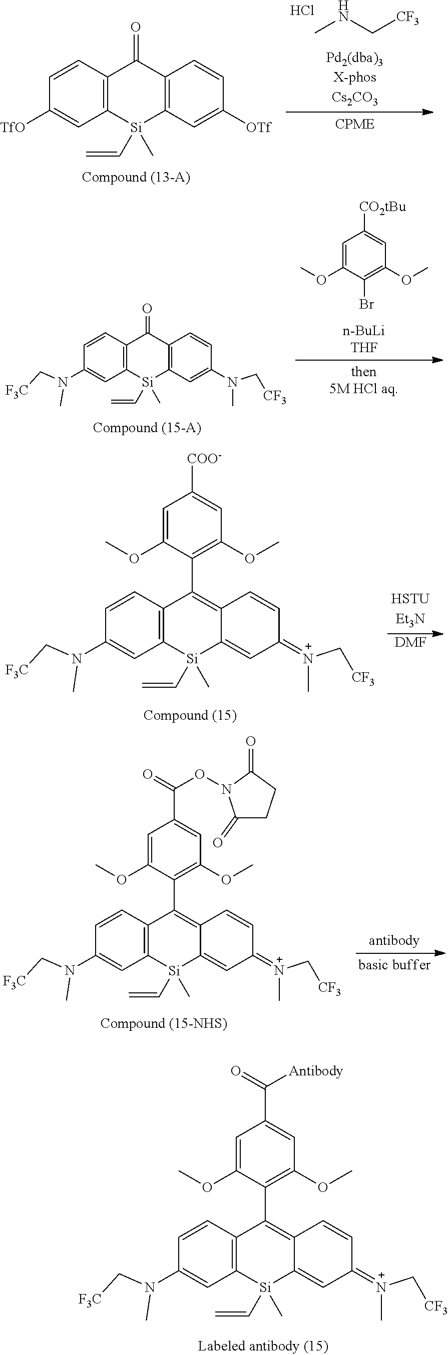

<Synthesis of Compound (15-A)>

A yellow powder compound (15-A) (45 mg, yield: 34%) was obtained in the same manner, except that in the synthesis method for the compound (13-B), methyl (2,2,2-trifluoroethyl)amine hydrochloride (manufactured by Matrix Scientific) was used instead of 3-fluoroazetidine hydrochloride.

[M+H⁺]⁺: 473

<Synthesis of Compound (15)>

A dark blue powder compound (15) (0.96 mg, yield: 1.6%) was obtained in the same manner, except that in the synthesis method for the compound (13), the compound (15-A) was used instead of the compound (13-B).

[M+H⁺]⁺: 637

<Synthesis of Compound (15-NHS)>

A dark blue powder compound (15-NHS) was obtained in the same manner, except that in the synthesis method for the compound (1-NHS), the compound (15) was used instead of the compound (1)

<Synthesis of Labeled Antibody (15)>

A labeled antibody (15) was obtained in the same manner except that in the synthesis method for the labeled antibody (1), the compound (15-NHS) was used instead of the compound (1-NHS).

### [Synthesis Example 16: Synthesis of Comparative Compound (1) and Comparative Labeled Antibody (1)]

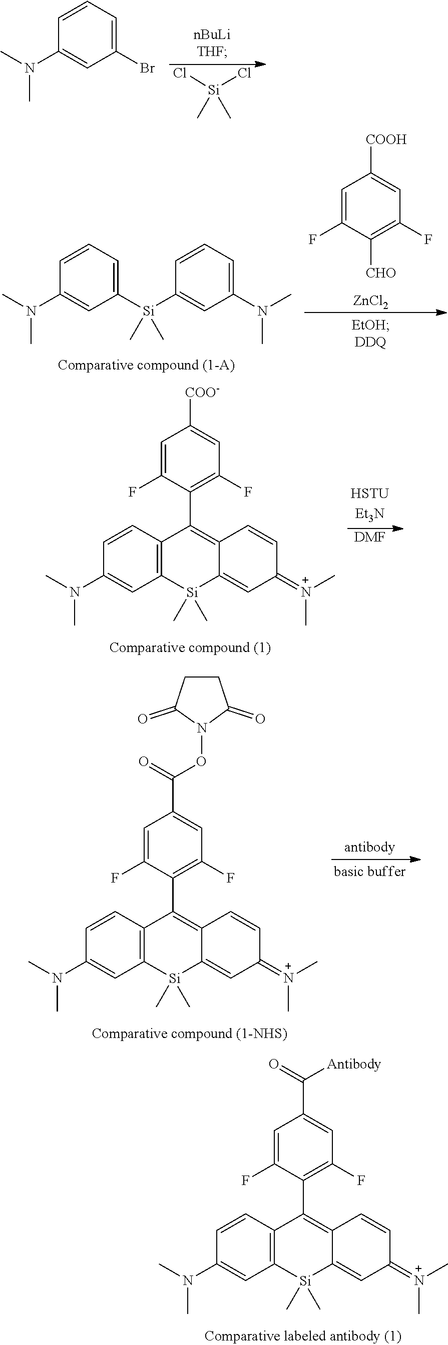

<Synthesis of Comparative Compound (1-A) to Comparative Compound (1-NHS)>

A comparative compound (1) (5 mg) and a comparative compound (1-NHS) (1 mg) were obtained in the same manner, except that in the synthesis method for the compound (1) and the compound (1-NHS), dichlorodimethylsilane (manufactured by Tokyo Chemical Industry Co., Ltd.) was used instead of dichloromethylvinylsilane.

Compound (11)

[M+H⁺]⁺: 465

Compound (11-NHS)

[M+H⁺]⁺: 563

<Synthesis of Comparative Labeled Antibody (1)>

A comparative labeled antibody (1) was obtained in the same manner except that in the synthesis method for the labeled antibody (1), the comparative compound (1-NHS) was used instead of the compound (1-NHS).

[1] Evaluation of Water Solubility

5 μL of a DMSO solution having a concentration of 20 mM of the above compound (hereinafter, referred to as a sample) and 495 μL of PBS having a pH of 7.4 were added and mixed in a 1.5 mL Eppendorf tube and stirred with a multi-shaker MS300 (product name, AS ONE Corporation) at 2,000 rpm for 30 minutes. The obtained mixed solution was allowed to be left for 60 minutes with light shielding and then subjected to centrifugal precipitation (12,000 rpm, 5 minutes). For the filtrate filtered using a filter having a pore diameter of 0.20 m, the sample concentration was measured with Nexera UHPLC [product name, manufactured by Shimadzu Corporation, column: Shim-pack XR-ODSII] (the sample concentration is 200 μM in a case where the sample is completely dissolved) and evaluated based on the following evaluation standards.

Regarding the water solubility, a high evaluation rank is highly preferable since the hydrophilicity is high, and in the present test, it is preferable that the water solubility satisfies the evaluation rank “C” or higher from the viewpoint of practicality as the fluorescent compound.

—Evaluation Standards for Water Solubility—
A: 100 μM or more
B: 10 μM or more and less than 100 μM
C: 1 μM or more and less than 10 μM
D: Less than 1 μM, or water solubility is low and thus concentration cannot be measured.

[2] Evaluation of Fluorescence Quantum Yield

A PBS solution (pH 7.4) of the above compound was evaluated according to the method described in the following reference document.

“A Guide to Recording Fluorescence Quantum Yields” (a HORIBA Scientific document, available from https://www.horiba.com/fileadmin/uploads/Scientific/Documents/Fluorescence/quantumyields trad.pdf)

Regarding the fluorescence quantum yield, a high evaluation rank is highly preferable since the fluorescence quantum yield is excellent, and in the present test, it is preferable that the fluorescence quantum yield satisfies the evaluation rank “C” or higher from the viewpoint of practicality as the fluorescent compound.

—Evaluation Standards for Fluorescence Quantum Yield—
A: 0.5 or more
B: 0.3 or more and less than 0.5
C: 0.1 or more and less than 0.3
D: less than 0.1
E: not evaluable due to low water solubility

[3] Evaluation of Maximum Molar Absorption Coefficient

A PBS solution (pH 7.4) of the above compound was evaluated according to the method described in the following reference document. The unit of the maximum molar absorption coefficient in the following evaluation standard is mol⁻¹ Lcm⁻¹.

“TECH TIP #6 Extinction Coefficients” (a document from Thermo Scientific, Inc., available from https://assets.thermofisher.com/TFS-Assets/LSG/Application-Notes/TR0006-Extinction-coeffi cients.pdf)

Regarding the maximum molar absorption coefficient, a high evaluation rank is highly preferable since the maximum molar absorption coefficient is excellent, and in the present test, it is preferable that the maximum molar absorption coefficient satisfies the evaluation rank “C” or higher from the viewpoint of practicality as the fluorescent compound.

—Evaluation Standard for Maximum Molar Absorption Coefficient—
A: 100,000 or more
B: 50,000 or more and less than 100,000
C: 10,000 or more and less than 50,000
D: less than 10,000
E: not evaluable due to low water solubility

[4] Evaluation of Light Resistance

The compound synthesized above was dissolved in a PBS solution (pH 7.4) so that the absorbance at the absorption peak wavelength was 0.095 to 0.105. In a state where this solution was exposed using a merry-go-round type light irradiator (xenon lamp UXL-500D-O manufactured by Ushio Inc., HA-50 filter, Y44 filter, exposure intensity: 22 mW/cm² (in terms of 500 nm)), the absorbance at the absorption peak wavelength of each compound was measured over time with a spectrometer (Agilent 8453, manufactured by Agilent Technologies, Inc.). The absorbance at the absorption peak wavelength before light exposure was set to 100% and the light exposure time until the absorbance at this absorption peak wavelength decreases by 20% (the absorbance at the absorption peak wavelength reaches 80%) was measured. The results were evaluated based on the following evaluation standards.

A high evaluation rank is highly preferable since stability is kept for a long time, and in the present test, it is preferable that the light resistance satisfies the evaluation rank “B” or higher from the viewpoint of practicality as the fluorescent compound.

—Evaluation Standards for Light Resistance—
A: 100 hours or more
B: 50 hours or more and less than 100 hours
C: 25 hours or more and less than 50 hours
D: 2 hours or more and less than 25 hours
E: Less than 2 hours
F: Not evaluable due to low water solubility

TABLE 1

| | | | | Maximum | |
|---|---|---|---|---|---|
| | | | Fluorescence | molar | |
| | Fluorescent | Water | quantum | absorption | Light |
| | compound | solubility | yield | coefficient | resistance |
| Example 1-1 | Compound (1) | C | C | A | A |
| Example 1-2 | Compound (2) | C | B | A | A |
| Example 1-3 | Compound (3) | C | C | A | A |
| Example 1-4 | Compound (4) | C | B | A | A |
| Example 1-5 | Compound (5) | C | D | A | A |
| Example 1-6 | Compound (6) | C | B | A | A |
| Example 1-7 | Compound (7) | C | B | A | A |
| Example 1-8 | Compound (8) | C | C | B | A |
| Example 1-9 | Compound (9) | B | B | A | A |
| Example 1-10 | Compound (10) | C | A | B | A |
| Example 1-11 | Compound (11) | C | A | C | A |
| Example 1-12 | Compound (12) | C | A | D | A |
| Example 1-13 | Compound (13) | C | A | B | — |
| Example 1-14 | Compound (14) | C | A | B | — |
| Example 1-15 | Compound (15) | C | A | C | — |
| Comparative | Comparative | C | C | A | A |
| Example 1-1 | compound (1) | | | | |
| Reference | AlexaFluor647 | A | B | A | D |
| Example 1-2 | | | | | |

The compounds (1) to (15) and the comparative compound (1) are respectively the compounds (1) to (15) and the comparative compound (1), which are synthesized above, and Alexa Fluor 647 is Alexa Fluor 647 (product name, product number: A33084) manufactured by Thermo Fisher Scientific, Inc.

In Example 1-13, Example 1-14, and Example 1-15, the light resistance cannot always be evaluated accurately and thus denoted as “-” in the above table. However, it has been confirmed that the compounds (13) to (15) tend to exhibit the same level of sufficient light resistance as the compounds (1) to (12).

From the results in Table 1 above, the following facts can be seen.

The commercially available fluorescent compound used in Reference Example 1-2 is inferior in light resistance.

On the other hand, in the compounds (1) to (15), which are the fluorescent compounds according to the embodiment of the present invention, and the comparative compound (1) light resistance is excellent, and all of water solubility, fluorescence quantum yield, and maximum molar absorption coefficient are in a preferred level from the viewpoint of practicality, and thus it is expected that a labeled biological substance exhibiting excellent light resistance and an excellent fluorescence quantum yield can be obtained.

Among these compounds, for example, the compound (6) in which at least one of R⁷, . . . , or R¹¹ in the fluorescent compound according to the embodiment of the present invention is a group including an electron withdrawing group in the fluorescent compound according to the embodiment of the present invention exhibits a better fluorescence quantum yield as compared with the compound (1) in which none of R⁷ to R¹¹ includes an electron withdrawing group. In addition, the compound (15) in which at least one of R⁷, . . . , or R¹¹ in the fluorescent compound according to the embodiment of the present invention is a group including an electron withdrawing group in the fluorescent compound according to the embodiment of the present invention exhibits a better fluorescence quantum yield as compared with the compound (4) in which none of R⁷ to R¹¹ includes an electron withdrawing group.

In addition, the compounds (10) to (14), in which in at least one of the combination of R⁷ and R⁸ or the combination of R¹⁰ and R¹¹ in the fluorescent compound according to the embodiment of the present invention, R⁷ and R⁸, or R¹⁰ and R¹¹ are bonded to each other to form a 4- to 7-membered aliphatic heterocyclic ring, exhibit a better fluorescence quantum yield as compared with the compound (9) in which both the combination of R⁷ and R⁸ and the combination of R¹⁰ and R¹¹ do not form an aliphatic heterocyclic ring having a 4- to 7-membered ring. Considering the fact that almost no change in the fluorescence quantum yield in association with the change in the structure of the substituent Z, which is described in J. Am. Chem. Soc. 2017, 139, 17397-17404, is observed, it is conceived that the effect that the fluorescence quantum yield is favorable although the compound (9) is different from the compounds (10) to (14) in the structure of the substituent Z in the fluorescent compound according to the embodiment of the present invention is an effect caused by the formation of the 4- to 7-membered aliphatic heterocyclic ring.

The present invention has been described together with the embodiments of the present invention. However, the inventors of the present invention do not intend to limit the present invention in any part of the details of the description unless otherwise designated, and it is conceived that the present invention should be broadly construed without departing from the spirit and scope of the invention shown in the attached “WHAT IS CLAIMED IS”.

What is claimed is:

1. A fluorescent compound represented by any one of General Formulae (I) to (IV) or a salt thereof, 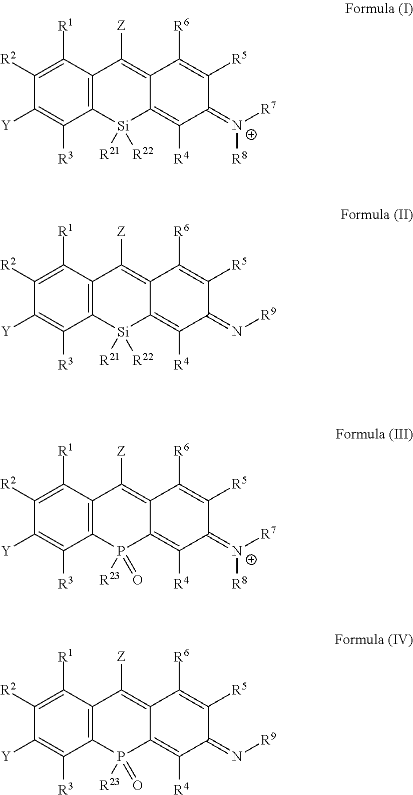
in the formulae, Y represents —NR¹⁰R¹¹ or —OR¹², and Z represents a group represented by Formula (A),
R¹ to R⁶ represent a halogen atom, a cyano group, or a group represented by Formula (A),
R⁷ to R¹² represent a group represented by Formula (A), where R⁷ and R⁸ may be bonded to each other to form a 4- to 7-membered aliphatic heterocyclic ring, R¹⁰ and R¹¹ may be bonded to each other to form a 4- to 7-membered aliphatic heterocyclic ring, and R⁷ to R¹¹¹ each may be bonded to adjacent R² to R⁵ to form a 5- to 7-membered aliphatic heterocyclic ring or aromatic heterocyclic ring,
R²¹ and R²² represent a group represented by Formula (A), and R²¹ and R²² may be bonded to each other to form a 4- to 7-membered aliphatic heterocyclic ring, provided that at least one of R²¹ or R²² is an alkenyl group, an alkynyl group, an aryl group, or a heteroaryl group, and
in a case where R²¹ is an aryl group and R²² is an alkyl group or an aryl group, in at least one of a combination of R⁷ and R⁸ or a combination of R¹⁰ and R¹¹, R⁷ and R⁸, or R¹⁰ and R¹¹ are bonded to each other to form a 4-membered aliphatic heterocyclic ring, and
R²³ represents a group represented by Formula (A),
*-L³-R¹¹¹ Formula (A)
in the formula, L³ is a single bond or a linking group obtained by combining one or two or more kinds among an alkylene group, an alkenylene group, an alkynylene group, an arylene group, a heteroarylene group, and each group represented by any one of Formula (1-1) to Formula (1-8),
R¹¹¹ represents a hydrogen atom, an alkyl group, an alkenyl group, an alkynyl group, an aryl group, a heteroaryl group, or a monovalent aliphatic heterocyclic group,
provided that in a case where a hydrogen atom as R¹¹¹ is a dissociative hydrogen atom, the dissociative hydrogen atom may be dissociated, and
* represents a bonding portion, 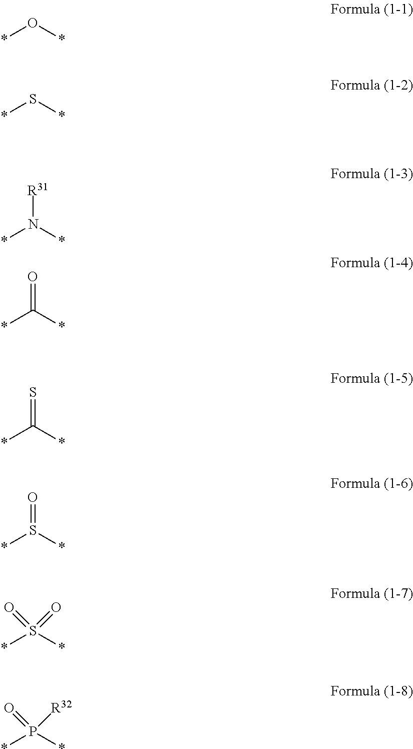
in the formulae, R³¹ and R³² represent a hydrogen atom or a substituent, and
* represents a bonding portion,
provided that the compound represented by Formula (I) or (II) or a salt thereof satisfies the following condition α, and the compound represented by Formula (III) or (IV) or a salt thereof satisfies the following condition β,
(Condition α)
in a case where Z is an aryl group, the aryl group is a group represented by Formula (C), 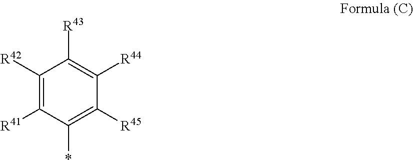
in the formula, R⁴¹ to R⁴⁵ represent a hydrogen atom or a substituent, and
* represents a bonding portion,
provided that at least one of (C-1), (C-2), or (C-3) is satisfied,
(C-1) at least one of R⁴², R⁴³, or R⁴⁴ is a group having any of a carboxy group or a substituent capable of being bonded to a biological substance,
(C-2) R⁴¹ and R⁴⁵ are an alkyl group or a halogen atom,
(C-3) at least one of R⁴¹ or R⁴⁵ is a fluorine atom or an alkoxy group,
(Condition β)
in at least one of a combination of R⁷ and R⁸ or a combination of R¹⁰ and R¹¹, R⁷ and R⁸, or R¹⁰ and R¹¹ are bonded to each other to form a 4-membered aliphatic heterocyclic ring.

2. The fluorescent compound or a salt thereof according to claim 1,
wherein the fluorescent compound or a salt thereof is represented by General Formula (I) or (II).

3. The fluorescent compound or a salt thereof according to claim 2,
wherein in at least one of the combination of R⁷ and R⁸ or the combination of R¹⁰ and R¹¹, R⁷ and R⁸, or R¹⁰ and R¹¹ are bonded to each other to form a 4- to 7-membered aliphatic heterocyclic ring,
provided that in a case where R²¹ is an aryl group and R²² is an alkyl group or an aryl group, the ring formed by bonding R⁷ and R⁸, or R¹⁰ and R¹¹ to each other in at least one of the combination of R⁷ and R⁸ or the combination of R¹⁰ and R¹¹ is a 4-membered aliphatic heterocyclic ring.

4. The fluorescent compound or a salt thereof according to claim 2,
wherein at least one of R⁷, . . . , or R¹¹ is a group including an electron withdrawing group.

5. The fluorescent compound or a salt thereof according to claim 2,
wherein Z is a group represented by Formula (C).

6. The fluorescent compound or a salt thereof according to claim 2,
wherein Y is —NR¹⁰R¹¹.

7. The fluorescent compound or a salt thereof according to claim 2,
wherein the fluorescent compound or a salt thereof is represented by General Formula (IA) or (IIA), 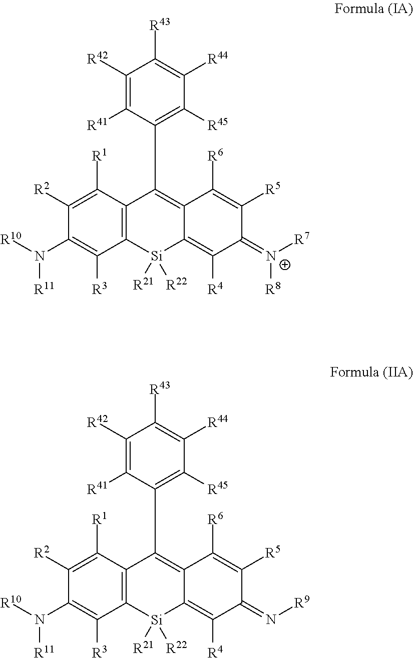
in the formulae, R¹ to R¹¹ and R⁴¹ to R⁴⁵ respectively have the same meanings as R¹ to R¹¹ and R⁴¹ to R⁴⁵,
R²¹ and R²² represent the group represented by Formula (A), and R²¹ and R²² may be bonded to each other to form a 4- to 7-membered aliphatic heterocyclic ring,
provided that at least one of R²¹ or R²² is an alkenyl group or an aryl group, and
in a case where R²¹ is an aryl group and R²² is an alkyl group or an aryl group, in at least one of the combination of R⁷ and R⁸ or the combination of R¹⁰ and R¹¹, R⁷ and R⁸, or R¹⁰ and R¹¹ are bonded to each other to form a 4-membered aliphatic heterocyclic ring.

8. The fluorescent compound or a salt thereof according to claim 7,
wherein the fluorescent compound or a salt thereof is represented by General Formula (IA),
R⁷, R⁸, R¹⁰, and R¹¹ are a hydrogen atom or an alkyl group having 1 to 3 carbon atoms, and
R⁴¹ to R⁴⁵ satisfy (C-1) and (C-2), satisfy (C-1) and (C-3), or satisfy all of (C-1) to (C-3) in the condition α.

9. The fluorescent compound or a salt thereof according to claim 7,
wherein the fluorescent compound or a salt thereof is represented by General Formula (IA),
in at least one of the combination of R⁷ and R⁸ or the combination of R¹⁰ and R¹¹, R⁷ and R¹, or R¹⁰ and R¹¹ are bonded to each other to form a 4- to 7-membered aliphatic heterocyclic ring, and
R⁴¹ to R⁴⁵ satisfy (C-1) and (C-2), satisfy (C-1) and (C-3), or satisfy all of (C-1) to (C-3) in the condition α.

10. The fluorescent compound or a salt thereof according to claim 1,
wherein the fluorescent compound or a salt thereof is represented by General Formula (III) or (IV).

11. The fluorescent compound or a salt thereof according to claim 10,
wherein at least one of R⁷, . . . , or R¹¹ is a group including an electron withdrawing group.

12. The fluorescent compound or a salt thereof according to claim 10,
wherein R²³ is a hydroxy group, an alkoxy group, an aryl group, or an alkyl group,
provided that in a case where R²³ is a hydroxy group, a hydrogen atom in the hydroxy group may be dissociated.

13. A fluorescently labeled biological substance that is obtained by bonding a biological substance to the fluorescent compound or a salt thereof according to claim 1.

14. The fluorescently labeled biological substance according to claim 13,
wherein the biological substance is any one of a protein, an amino acid, a nucleic acid, a sugar chain, or a lipid.

15. The fluorescently labeled biological substance according to claim 13,
wherein the bonding between the fluorescent compound or a salt thereof and the biological substance is a bonding formed by any one of the following i) to v),
i) a non-covalent bond or covalent bond between peptides,
ii) a Van der Waals interaction between a long-chain alkyl group in a fluorescent compound and a lipid bilayer or lipid in a biological substance,
iii) an amide bond formed by reacting an N-hydroxysuccinimide ester in a fluorescent compound with an amino group in a biological substance,
iv) a thioether bond formed by reacting a maleimide group in a fluorescent compound with a sulfanyl group in a biological substance,
v) a bond associated with a formation of a triazole ring, which is formed by a Click reaction between an azido group in a fluorescent compound and an acetylene group in a biological substance, or between an acetylene group in a fluorescent compound and an azido group in a biological substance.
